(19) United States
(12) Patent Application Publication
(10) Pub. No.: US 2013/0150418 A1
(43) Pub. Date: Jun. 13, 2013

(54) APPARATUS AND METHOD FOR THE TREATMENT OF ABNORMAL UTERINE BLEEDING

(71) Applicants: Steven D. Goedeke, Forest Lake, MN (US); Charles L. Euteneuer, St. Michael, MN (US); Thomas K. Hektner, Medina, MN (US); David M. Crompton, Shoreview, MN (US)

(72) Inventors: Steven D. Goedeke, Forest Lake, MN (US); Charles L. Euteneuer, St. Michael, MN (US); Thomas K. Hektner, Medina, MN (US); David M. Crompton, Shoreview, MN (US)

(21) Appl. No.: 13/712,471

(22) Filed: Dec. 12, 2012

Related U.S. Application Data
(60) Provisional application No. 61/569,986, filed on Dec. 13, 2011.
(60) Provisional application No. 61/569,978, filed on Dec. 13, 2011.

(51) Int. Cl. A61K 31/4174; A61F 6/22

(52) U.S. Cl. CPC A61K 31/4174; A61F 6/225
USPC 514/401; 128/830

(57) ABSTRACT

Method and apparatus are disclosed for applying a therapeutic amount of a non-systemic vasoconstrictor inside the uterus to control abnormal uterine bleeding. The abnormal bleeding can be due to excessive menstrual blood flow, bleeding from a surgical procedure, postpartum bleeding or any other acute or chronic condition. The vasoconstrictor includes topical agents such as an alpha-adrenergic agonist, for example oxymetazoline. The delivery system can include a catheter having means for retaining position of a distal portion within the uterus. A proximal portion can extend outside of the body for coupling to a vasoconstrictor source, or alternatively, the proximal portion can terminate within the vaginal canal and include a docking port for coupling to a source of vasoconstrictor that is inserted therein. In other embodiments, an applicator is disclosed that is positioned in fluid communication with the lumen of the cervix and allows application of a vasoconstrictor therein.

### FIELD OF THE INVENTION

The present invention relates generally to the field of gynecological medicine. More particularly, the present invention relates to methods and apparatus for treatment of abnormal or heavy uterine bleeding by delivering a therapeutic amount of a non-systemic vasoconstrictor into the uterus.

### BACKGROUND

Abnormal uterine bleeding (AUB) has a significant impact on the life of many women. According to Marret, AUB accounts for up to 20% of visits to the gynecologist yet the causes and mechanisms of such dysfunction are less than clear. Marret, H., “Clinical Practice Guidelines on Menorrhagia”, European Journal of Obstetrics & Gynecology and Reproductive Biology 152 (2010) 133-137. Further, AUB is a frequently cited indication for hysterectomy and accounts for as many as 25% of all hysterectomies. Another study listed AUB as the main presenting problem in at least half of all the hysterectomies reported. Liu, “Systematic Review”, Value in Health, Vol. 10, No. 3 (2007) 183-194.

Blood is supplied to the uterus and its endometrium by the ovarian and uterine arteries which enter the wall of the uterus and give rise to arcuate arteries in the myometrium. Radial arteries branch from the arcuate arteries. Basal arteries branch from the radial arteries and cross the mometrial endometrial junction and supply blood to the basal endometrium via the spiral arterioles. Spiral arterioles run toward the endometrial surface or functional layer of the endometrium. The spiral arterioles give rise to the capillaries which form a plexus in the subepithelium of the endometrium. As a woman goes through the pre-ovulatory phase of the cycle, the length of the spiral arterioles increase five-fold, leading to coiling. Pre-menstrually, the endometrium regresses, and the spiral arterioles continue to coil. Just before the start of menses, blood flow slows in the spiral arteries due to vasoconstriction which appears to be followed by dilation of the arterioles and the onset of bleeding.

Abnormal and normal uterine bleeding are defined in terms of the regularity, frequency, duration and volume of menstrual bleeding. Although terminology varies, one accepted standard based on volume is that abnormal uterine bleeding is the loss of greater than 80 ml. of blood per menstruation. Heavy menstrual bleeding can also be defined in terms of frequency and duration of menstruation.

There can be many causes of abnormal or heavy uterine bleeding, some uterine causes and some systemic causes. Uterine causes can include fibroids, endometrial polyps, endometriosis and pelvic inflammatory disease. Systemic causes can include coagulation disorders and clotting factor deficiencies as well as hypothyroidism. However, about half of women with abnormal or heavy uterine bleeding have no anatomical or endocrinological abnormality that can be detected.

Current treatments can include endocrine based approaches. For example, some patients show a reduction in bleeding when taking oral contraceptives. Further, a levonorgesterel-releasing intrauterine device for birth control has shown a reduction in menstrual blood flow in women who have the IUD device implanted. However, both of these treatments can have side effects.

Other treatments can include a hysterectomy and endometrial ablation. However, both of these approaches will prevent the possibility of future pregnancy. Further, a hysterectomy is a major surgery and loss of the organ will require future hormonal drug therapy with attendant side effects.

There is a need for an alternative treatment option for abnormal or heavy menstrual bleeding. In particular, a treatment that does not require surgery or hormone therapy is preferred. Further, for many, the option of future pregnancy should be maintained with use of this alternative treatment. Finally, the alternative treatment should be free of detrimental side effects or risks that are present with current endocrine based treatment approaches.

### BRIEF SUMMARY

The present disclosure is directed to the application of a therapeutic amount of a non-systemic vasoconstrictor to the inside of the uterus. The vasoconstrictor can be a topical vasoconstrictor. Further the vasoconstrictor can be non-hormonal and non-steroidal. This can include an alpha-adrenergic agonist which can activate both alpha-1 and -2 receptors. One drug can include oxymetazoline or OMZ. Application inside the uterus can include application to the endometrial layer prior to and or during menorrhagia, which can be defined as excessive blood loss of greater than 80 ml. per menstruation. Heavy uterine bleeding can also be defined as frequent or long duration menstruation. Alternatively, the therapeutic amount of non-systemic vasoconstrictor can be applied prior to menstruation in individuals having a history of excessive blood loss during menstruation. The therapeutic amount can also be applied in other acute incidents of excessive uterine bleeding, as in post-surgery or postpartum situations that may arise.

The therapeutic amount of a non-systemic vasoconstrictor can be delivered internal to the uterus in a water-based solution or saline solution. Other carriers can also be used, such as gels. The gels may regulate the rate of release of the vasoconstrictor to the uterine wall and extend the period of treatment relative to a saline carrier. For example, the therapeutic amount may be dispensed by the gel over a 72 hour period which could be equivalent to three daily doses. A combination of instant and time release medication can also be administered so that an initial bolus of vasoconstrictor causes immediate reduction in bleeding when placed in the uterus while the time release portion continues to maintain the control of bleeding over an extended period of time.

In one method of treatment, a therapeutic amount of a topical vasoconstrictor is applied inside the uterus. The therapeutic amount of topical vasoconstrictor can be about 0.75 mg. to about 3 mg. Further, the topical vasoconstrictor can be an alpha-adrenergic agonist such as oxymetazoline. The topical vasoconstrictor can include a water-based carrier such as saline which dissolves the vasoconstrictor. A therapeutic amount of the solution can include about 1 cc. to about 5 cc. of solution having a concentration of about 0.02% to about 0.08% by weight of topical vasoconstrictor.

The vasoconstrictor can be administered into the uterus using a delivery system. The delivery system can include a catheter having a distal portion, a proximal portion, and a lumen therethrough, the distal portion sized for disposition through the uterine cervix into the uterus with the proximal portion extending at least into the vaginal canal. The delivery system can also include means for retaining the distal portion in position within the uterus. For example, the means for retaining the distal portion in position within the uterus can include a distal portion having a t-shaped distal end. Alternatively, the means for retaining the distal portion in position within the uterus can include a suture attachment to the vaginal wall on the proximal portion of the catheter. In another embodiment, the means for retaining the distal portion in position within the uterus can include a ring connected to the proximal portion of the catheter for disposition and retention in the fornix area. This ring could also be a coiled portion of the catheter disposed in the fornix area.

The proximal portion of the delivery system can terminate within the vaginal canal and include a docking port on the proximal end thereof for receiving a distal end of an injection assembly in fluid communication with the lumen therein. The docking portion can include a penetrable septum for access to the lumen of the delivery system. Further, the docking port can include a funnel-shaped receptacle for receiving the injection assembly.

In other embodiments, the vasoconstrictor can be delivered with a cervical positioning applicator assembly. In these embodiments, a catheter into the uterus through the cervix is not needed. The cervical positioning applicator can include a shaft having a distal portion including an expandable positioning ring, the expandable positioning ring can assume a first unexpanded profile for insertion in the vaginal canal and a second expanded profile generally in contact with the fornix area in the upper vaginal canal, which in the second expanded profile positions a central axis of the ring over the uterine cervix. The applicator can further include an injection port having a distal projection located in the central portion of the ring that fluidly couples with the lumen of the cervix for injection of a vasoconstrictor therein. The injection port can include an inflatable membrane having a first retracted position and upon inflation of the membrane moves to a second inflated position with the injection port moving along the central axis of the positioning ring into generally sealing contact with the cervix.

The shaft can include multiple reservoirs and a plunger assembly, wherein a first reservoir includes an inflation fluid for expanding the positioning ring and a second reservoir includes a vasoconstrictor. The plunger assembly can include a first push rod in fluid communication with the first reservoir and a second push rod in fluid communication with the second reservoir. A proximally extending actuator slidably disposed within the shaft can be included, wherein distal movement of the actuator first contacts the first push rod to force fluid to deploy the positioning ring and then contacts the second push rod to eject the vasoconstrictor.

In some further embodiments, the applicator can include a retractable sheath disposed about at least the distal portion of the shaft. The positioning ring can include a self-expanding o-ring retained in an unexpanded profile by the sheath and deployed to an expanded profile by retraction of the sheath.

In another further embodiment of an applicator, the cervical positioning applicator can include a shaft having a distal portion carrying an expandable sponge or foam member. The sponge or foam member can include selected foam portions of preferred density and cell structure which either hold the vasoconstrictor until pressure is applied to squeeze it therefrom or in the alternative portions can have a closed cell or relatively non-porous structure which directs the vasoconstrictor toward the cervix and cervical opening when the foam member has pressure applied to it or is otherwise squeezed. The expandable foam member can assume a first unexpanded profile, as retained within an applicator sleeve, for insertion in the vaginal canal and a second expanded profile generally in contact with the cervix and/or fornix area in the upper vaginal canal. In the second expanded profile at least a portion of the foam member lies over the cervical opening and at least a portion of the cervix. In a preferred embodiment, open celled porous foam will face the cervical opening in the second position and this foam will be generally saturated with vasoconstrictor material. Mechanical pressure on the foam squeezes the vasoconstrictor out of the foam into the cervical opening and further into the uterus. Mechanical pressure can be asserted on the foam by its expansion against the opposing vaginal wall. Alternatively, the distal portion of the applicator can an inflatable membrane having a first retracted position and upon inflation of the membrane moves to a second inflated position which puts upward pressure on the foam member forcing the vasoconstrictor into the cervical canal and into the uterus.

In one embodiment of a foam member applicator, the vasoconstrictor is pre-loaded into the foam. In an alternative embodiment, the vasoconstrictor is contained in a reservoir either separated from or with the compressed foam. In this alternative embodiment, the foam draws in the vasoconstrictor as it expands so that is generally saturated when expanded, much as a compressed sponge absorbs liquid as it expands.

The above summary of some embodiments is not intended to describe each disclosed embodiment or every implementation of the present invention. The Figures and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1A is a schematic side cross sectional view of the pelvic region depicting a vasoconstrictor delivery device including a catheter distal portion extending into the uterus or uterine cavity;

FIG. 1B is a schematic frontal cross sectional view of the pelvic region depicting the delivery system of FIG. 1A having a distal T-shaped section to aid in retention within the uterus and distribute a vasoconstrictor therefrom;

FIG. 2A is a schematic side cross sectional view of the pelvic region depicting an alternative embodiment having a ring structure positioned within the fornix area for retaining catheter position and can include a reservoir of fluid;

FIG. 2B is a more detailed view of the ring of FIG. 2A as positioned in the fornix area;

FIG. 3A is a schematic side cross sectional view of the pelvic region depicting an alternative embodiment having a coiled catheter structure positioned within the fornix area for retaining catheter position and can allow withdrawal of the proximal end of the catheter from the vagina for injection of a vasoconstrictor and subsequent re-coiling back to the fornix area;

FIG. 3B is schematic frontal cross sectional view of the pelvic region depicting the delivery system of FIG. 3A having a coil section positioned in the fornix area;

FIG. 3C is a more detailed view of the coil of FIG. 3A as positioned in the fornix area;

FIG. 3D is a more detailed view of the coil of FIG. 3B as positioned in the fornix area;

FIG. 4A is a schematic side cross sectional view of the pelvic region depicting a vasoconstrictor delivery device including a catheter distal portion extending into the uterus or uterine cavity and suture attachment in the vaginal wall for catheter retention;

FIG. 4B is a more detailed view of the suture attachment of FIG. 4A;

FIG. 5A is a schematic side cross sectional view of the pelvic region depicting an alternative embodiment including a catheter terminating proximally within the vaginal canal and including a suture attachment;

FIG. 5B is a more detailed view of the delivery system of FIG. 5A depicting a proximal docking port on the catheter;

FIG. 6A is partial cross sectional view of an alternative embodiment illustrating the combination of a ring structure for retention of a distal catheter portion and a docking port located on the ring structure for coupling to a source of vasoconstrictor;

FIG. 6B is a closer view of the positioned ring structure of FIG. 6A illustrating that the ring can have a lumen in fluid communication with the distal portion of the catheter;

FIG. 6C is a closer view of the distal end of the catheter of FIG. 6A illustrating that the distal end can include a ball-like end having a port for distribution of the vasoconstrictor in the uterus;

FIG. 6D is a closer view of the docking port of FIG. 6A illustrating the inclusion of a septum for receiving a source of vasoconstrictor therethrough;

FIG. 6E depicts a source of vasoconstrictor docked in fluid communication with the septum of FIG. 6D having a needle portion piercing the septum;

FIG. 7A is a schematic frontal cross sectional view of the pelvic region depicting an alternative delivery system having a retention ring and a docking port located in alignment with the cervical canal;

FIG. 7B is a more detailed view depicting the retention ring and docking port of FIG. 7A;

FIG. 7C is schematic view of an applicator docking with the port of the delivery system of FIG. 7A;

FIG. 7D is a partial perspective view of a distal portion of the applicator of FIG. 7C illustrating a partial cup-like portion that contacts the fornix area for positioning the applicator in relation to the docking port;

FIG. 7E is a schematic partial plan view of a distal docking portion of the applicator that interfaces with the port on the delivery system of FIG. 7B;

FIG. 8A-8D are schematic cross sectional views of applicators that can interface with docking ports of the various embodiments of delivery systems disclosed including provision for a cleansing solution prior to injection of the vasoconstrictor;

FIG. 9A-9D are schematic cross sectional views of an alternative applicator system that includes recycling of a cleansing fluid that cleans a septum area of the delivery system port prior to injection of the vasoconstrictor;

FIG. 10A-10D are schematic cross sectional views illustrating a multi-reservoir applicator that can allow for staged injection of various media by movement of a single plunger;

FIG. 11A is a schematic perspective view of an alternative vasoconstrictor applicator designed to mate directly with the cervical canal and does not require a catheter to access the uterus;

FIG. 11B is a schematic perspective view of the applicator of FIG. 11A with the outer sheath removed illustrating the mechanism for staged deployment of the applicator with distal movement of the plunger;

FIG. 11C is a schematic perspective view of the applicator of 11A with a cervical positioning ring deployed;

FIG. 11D is a schematic perspective view of the applicator of FIG. 11B with the outer sheath removed illustrating distal plunger movement to deploy the cervical positioning ring;

FIG. 11E is a schematic perspective view of the applicator of FIG. 11C having an injector assembly deployed within the positioning ring;

FIG. 11F is a schematic perspective view of the applicator of FIG. 11E with the sheath removed illustrating distal plunger movement to inject vasoconstrictor through the injection assembly when mated with the cervical canal;

FIG. 12A is a schematic view of an alternative vasoconstrictor applicator that incorporates a foam member within a sheath assembly that is deployed via an inflatable member to mate with the cervical opening;

FIG. 12B is schematic view of the vasoconstrictor applicator of FIG. 12A illustrating the foam member in a deployed state for mating to the cervical opening;

FIG. 12C is a schematic view of another alternative vasoconstrictor applicator having a distal foam member retained within a sheath for insertion in the vaginal canal;

FIG. 12D is a schematic view of the vasoconstrictor applicator of FIG. 12C with the distal foam member depoloyed wherein expansion of the foam cause mechanical force to discharge the vasoconstrictor from the foam; and,

FIG. 12E is a schematic view of an alternative distal foam structure incorporating closed and open cell foam that directs the flow of vasoconstrictor when force is applied to the foam.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF A PREFERRED EMBODIMENT OF THE INVENTION

### Nomenclature

10 Pelvic Region
12 Uterus
14 Bladder
16 Colon
20 Vaginal Canal
22 Fornix Area
24 Uterine Cervix
26 External Ostium
28 Uterine Cavity
30 Ovaries
32 Fallopian Tubes
100 Device
102 Distal Portion
104 Proximal Portion
106 Proximal End
110 T-Section
120 Ring Structure
121 Reservoir
123 Proximal Port
122 Arms
124 Junction
130 Proximal Portion
150 Suture Attachment
154 Docking Port
157 Septum
159 Distal Portion
161 Applicator
163 Distal Projection
200 Delivery System
202 Retention Ring
204 Docking Port
206 Cervical Canal
208 Funnel Shaped Receptacle
215 Applicator Device
217 Distal End
219 Cup-Like Portion
300 Applicator
302 Syringe
304 Chamber
306 Plunger
308 Discharge Port
310 Lumen
312 Coupling
314 First Internal Chambers
316 Second Internal Chambers
318 Distal Seal
320 Proximal Wall
321 Second Walls
402 Syringe
404 Chamber
406 Plunger
408 Discharge Port
410 Lumen
412 Nozzle
414 First Chamber
415 Septum
416 Second Chamber
418 Distal Seal
425 Tube
426 Proximal Side
430 Needle
450 Applicator
452 Valve
454 First Syringe
456 Second Syringe
458 Syringe
460 Plunger
462 Proximal Discharge Port
464 Distal Discharge Port
500 Applicator Assembly
502 Shaft
504 Plunger Assembly
506 Distal Portion
508 Expandable Ring
510 Reservoir Housing
512 First Push Rod
514 Second Push Rod
516 Injection Assembly
518 Injection Port
520 Injection Port
600 Applicator Assembly
602 Sheath Portion of Shaft
604 Shaft
610 Upper Surface of Foam Member
612 Perimeter Portion of Foam Member
614 Central Portion of Foam Member
616 Balloon Member
618 Window
620 Expandable Foam Member
650 Applicator Assembly
652 Shaft
654 Sheath Portion of Shaft
656 Shaft
660 Expandable Foam Member
662 Perimeter Portion of Foam Member
664 Central Portion of Foam Member

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the claims, the singular forms “a”, “an”, and “the” include plural referents unless the content clearly dictates otherwise. As used in this specification and the claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

The present disclosure is directed to methods and apparatus for treatment of abnormal uterine bleeding or any other undesirable uterine bleeding. Abnormal uterine bleeding (AUB) is generally defined in terms of the menstrual cycle, to include a blood loss of greater than 80 ml. in a menstrual cycle. It can also be defined in terms of frequency and duration of menstruation. Although the present disclosure is primarily directed to treatment of AUB and generally discussed in that context, the method and apparatus are also useful for treating any undesirable uterine bleeding related and/or unrelated to menstruation. For example, post-surgery bleeding that may be associated with treating a uterine fibroid, or post-partum bleeding can be present after delivery of a child, or bleeding could be due to blood thinners in a patient's system. Therefore, for purposes of this disclosure, AUB is considered to include the various causes of undesirable bleeding from the uterus.

The present disclosure is directed to the application of a therapeutic amount of a non-systemic vasoconstrictor to the inside of the uterus. The vasoconstrictor can be a topical vasoconstrictor. Further, the vasoconstrictor can be non-hormonal and non-steroidal. This can include an alpha-adrenergic agonist which can activate both alpha-1 and -2 receptors. One drug can include oxymetazoline or OMZ. Application inside the uterus can include application to the endometrial layer prior to and or during menorrhagia.

The vasoconstrictor is used to normalize uterine bleeding, not necessarily to completely stop bleeding as such what constitutes the application of a therapeutic amount of a topical vasoconstrictor inside the uterus can vary from patient to patient and also with each menstrual cycle of that patient. In some patients the therapeutic amount of topical vasoconstrictor is about 0.75 mg. to about 3 mg. in each daily cumulative dose, whether applied in a single bolus, or over a period of hours. In others, the daily dose is about 1 mg. to about 2 mg. daily, while in others the dose is about 1.5 mg. daily. The vasoconstrictor may be dissolved in a water-based carrier, such as saline, and can include a therapeutic amount of about 1 cc. to about 5 cc. having a concentration of about 0.02% to about 0.08% of topical vasoconstrictor. Alternatively, the dose can be about 2 cc. to about 4 cc. of the same concentration.

The vasoconstrictor can be placed in a controlled release carrier that forms a pellet or suppository structure that can be inserted in the uterus. The pellet can include enough vasoconstrictor to provide treatment through the bleeding of one menstrual period and be inserted at or just prior to that cycle. Alternatively, the pellet could include enough vasoconstrictor for multiple cycles and be slow released for several months, including release during the time between menstrual cycles. To avoid treatment during non-bleeding, the pellet could be designed with an active release mechanism that opens dose containing compartments within the pellet in response to a signal from a controller. This type of dose delivery would use a remotely burned or eroded window on a dosing compartment. Alternatively, the doses could be encapsulated in differing carriers, such as biodegradable polymers of differing breakdown characteristics so that dosing occurs approximately on a monthly cycle. Different polymers can be used, or the same polymer having more or less cross linking can be used to vary the time of release. With this embodiment, the entire pellet or suppository can be bioresorbable.

Alternatively, the vasoconstrictor could be carried by a known intra-uterine device (IUD) and have a slow release carrier that allows release of the vasoconstrictor over a period of several months. Currently, some IUDs include a progesterone material in a carrier to provide effective birth control. The vasoconstrictor could be added with the progesterone or replace it in some alternative embodiments.

The vasoconstrictor can be applied to the interior of the uterus using any of many delivery apparatus. Some representative embodiments of delivery systems are included herein. The delivery system can be a catheter-based system that includes a distal portion that extends into the uterus through the cervical canal. As disclosed herein, some catheter systems include a portion that remains within the body between doses and provides subsequent access to this portion of the delivery system during subsequent treatments. Alternatively, a catheter based system can be inserted with each treatment, including insertion of a distal portion into the uterus through the cervical canal. In other delivery systems, a catheter is not necessary as the delivery system can be designed to mate or couple with the cervix and opening into the cervical canal. When the delivery system is coupled, the vasoconstrictor can be injected or forced into the cervical canal that is in fluid communication with the inside of the uterus which allows injection therein. As with catheter systems, this type of injection system can include a docking station that is left inserted into the vaginal canal between doses for mating with an injection device on later doses. Alternatively, the injection system can include all components that are inserted with each dose being designed to position properly relative to the uterine opening and fluidly couple therewith for injection, followed by complete removal of the device between doses. The delivery system can include structure that manipulates the cervix to properly position the device at the cervical opening and/or can include structure that uses vaginal landmarks, such as the fornix, to properly position the delivery system relative to the cervix.

Multiple doses of a vasoconstrictor could be included in the devices of the present invention. For example, the components could include a reservoir of vasoconstrictor and a patient activated pump or delivery system that is in fluid communication with a distal portion of a catheter that is inserted into the uterus. The reservoir and pump could be positioned in the fornix area and left for several days during a menstrual cycle or several cycles. Alternatively, the pump can be exterior to the body and connected via a proximal portion of the catheter.

The treatment cycle can vary from patient to patient under various embodiments of the present disclosure. For example, a single daily dose to the uterus over a period of three days during menstrual bleeding may be sufficient. In other patients and treatment cycles, multiple daily doses may be necessary to reduce or control bleeding. Yet in other patients or treatment cycles, a continuous slow release may best control bleeding. Combinations of these cycles may also be beneficial. For example, at the start of menstrual bleeding a single higher dose or bolus followed by smaller support doses may most effectively control bleeding.

Other therapeutic agents can be included with the vasoconstrictor. For example, some systems can include cleansing of the cervical area prior to fluid communication with the interior of the uterus. This can include antiseptic and antibacterial agents. Further, a pain reliever, such as a non-steroidal anti-inflammatory drug could be included with the vasoconstrictor to provide local pain relief, such as relief from menstrual cramping. Alternatively, a cleansing flush of the uterus may precede application of the vasoconstrictor to clean the endometrial area and allow better contact between the vasoconstrictor and open arterial vessels to be constricted.

In other alternative embodiments, the delivery system or portions thereof may be inserted by a medical professional while in other embodiments the patient may insert all or a portion of the system with each dose. This can include self-catheterization or insertion of a distal portion of a catheter into the uterus, in conjunction with a visual aid such as a videoscope.

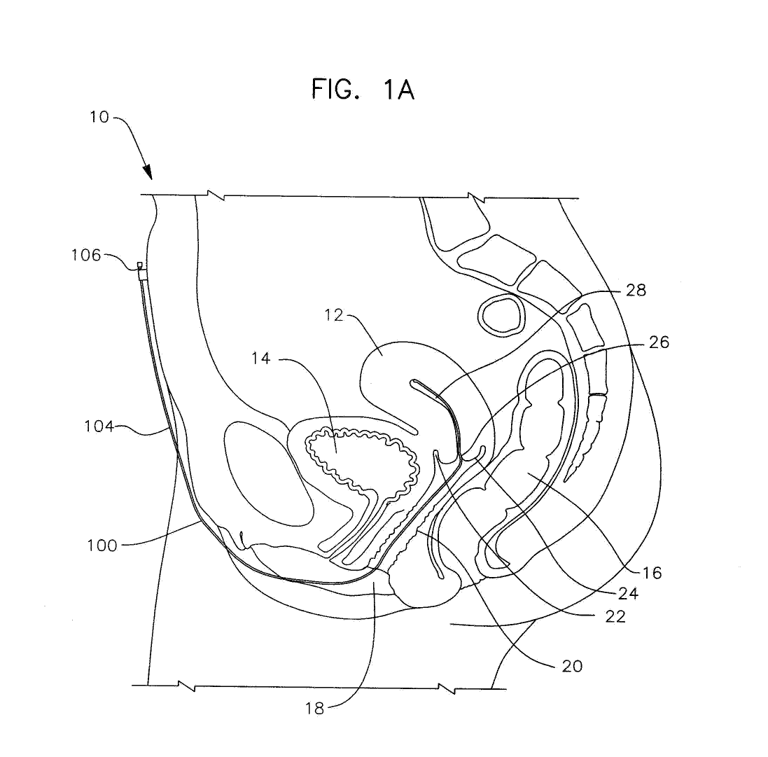

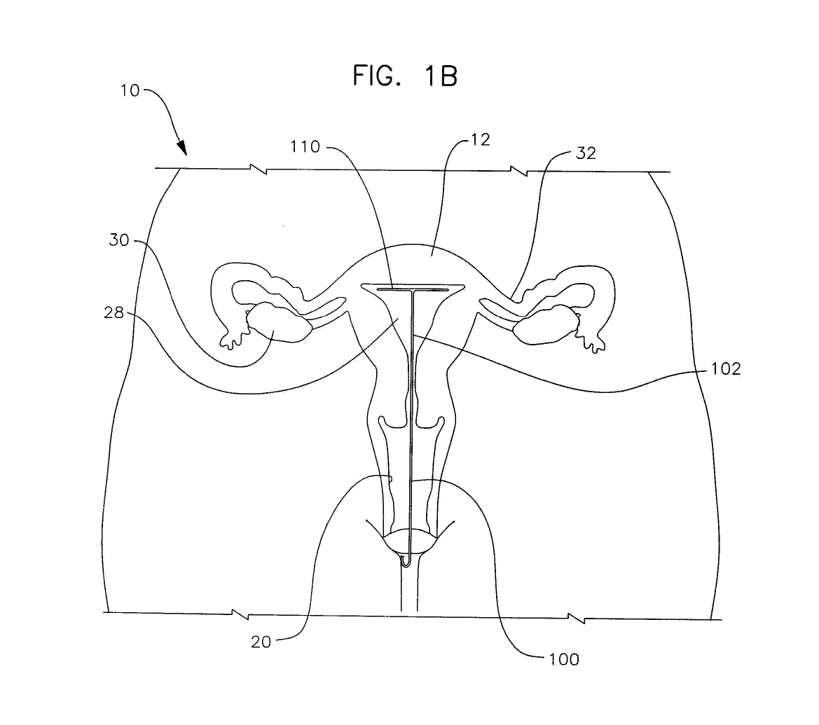

Now referring to FIG. 1A, a schematic side cross sectional view of the pelvic region 10 of a woman is depicted, including the uterus 12 as positioned relative to the bladder 14 and colon 16. The uterus 12 is illustrated in a non-pregnant state as a generally collapsed structure located at the distal end of the vaginal canal 20. The vaginal opening 18 provides access to the uterus 12 through the vaginal canal 20. At the juncture of the vaginal canal 20 and uterus 12 is the fornix area 22 which forms a generally circular recess having the uterine cervix 24 protruding from within the area defined by the generally circular boundary of the fornix area 22. Within a central portion of the uterine cervix 24 lies the external ostium 26 which provides access to the cervical canal to the internal ostium and then the inside or interior 28 of the uterus.

As depicted in FIG. 1A, a vasoconstrictor delivery device 100 is inserted into the vaginal canal 20 through the vaginal opening 18. The delivery device 100 includes a catheter distal portion 102 extending into the uterus 12 or uterine cavity 28. A proximal portion 104 of the delivery device 100 extends a sufficient distance proximally to have a proximal end 106 accessible on the exterior surface of the pelvis for injection of a vasoconstrictor therein. The proximal end 106 can include a septum or other type of isolatable connection for cooperation with an applicator or syringe when in use.

In FIG. 1B, a schematic frontal cross sectional view of the pelvic region depicting the delivery system of FIG. 1A is provided. Additionally depicted are the ovaries 30 and fallopian tubes 32 in relation to the uterus 12. The vasoconstrictor delivery system 100 is illustrated having a distal portion 102 residing on the interior 28 of the uterus 12. The distal portion 102 includes a t-section 110 proximate its distal end in some alternative embodiments. The t-section 110 can improve retention of the distal portion within the uterus and can aid in dispersion of the vasoconstrictor when injected into the uterine cavity therethrough.

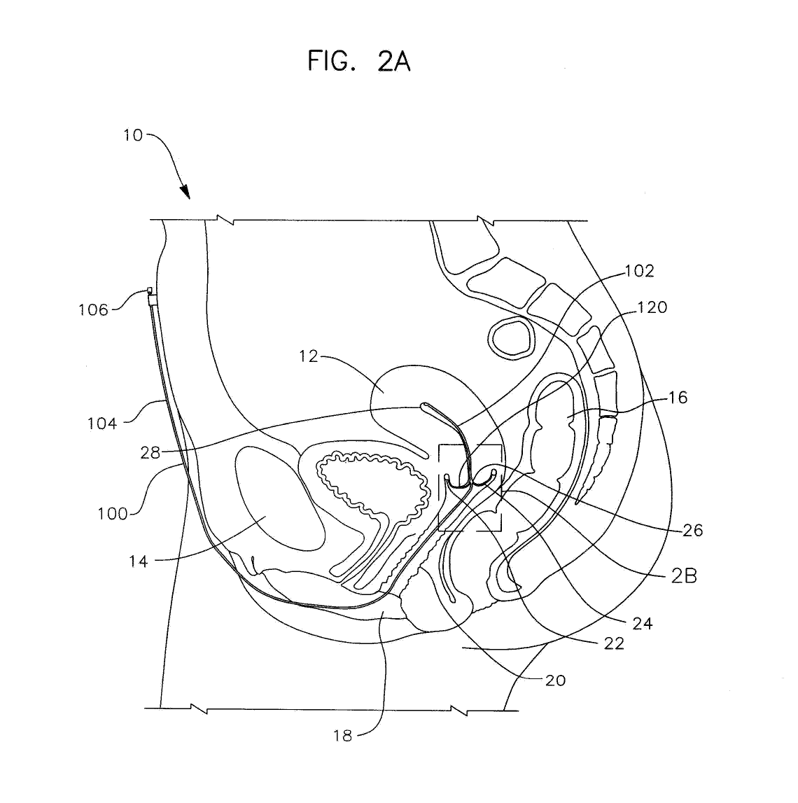

FIG. 2A is a schematic side cross sectional view of the pelvic region 10 depicting an alternative embodiment of the present disclosure. As with FIG. 1A, the uterus 12 is shown positioned relative to the bladder 14 and colon 16. The uterus 12 is illustrated in a non-pregnant state as a generally collapsed structure located at the distal end of the vaginal canal 20. The vaginal opening 18 provides access to the uterus 12 through the vaginal canal 20. At the juncture of the vaginal canal 20 and uterus 12 is the fornix area 22 which forms a generally circular recess having the uterine cervix 24 protruding from within the area defined by the generally circular boundary of the fornix area 22. Within a central portion of the uterine cervix 24 lies the external ostium 26 which provides access to the cervical canal to the internal ostium and then the inside or interior 28 of the uterus.

As depicted in FIG. 2A, a vasoconstrictor delivery device 100 is inserted into the vaginal canal 20 through the vaginal opening 18. The delivery device 100 includes a catheter distal portion 102 extending into the uterus 12 or uterine cavity 28. A proximal portion 104 of the delivery device 100 extends a sufficient distance proximally to have a proximal end 106 accessible on the exterior surface of the pelvis for injection of a vasoconstrictor therein. The proximal end 106 can include a septum or other type of isolatable connection for cooperation with an applicator or syringe when in use. Additionally, the embodiment of FIG. 2A includes a ring structure 120 positioned within the fornix area 22 for retaining catheter position and can include a reservoir of fluid for injection into the uterine cavity 28.

The catheter distal portion and/or catheter proximal portion can be affixed to the ring structure 120. The relationship is better illustrated in FIG. 2B which shows the ring structure 120 having arms 122 that connect to the catheter portion at a junction 124. The ring structure 120 resides in the fornix area 22, taking advantage of the recessed anatomical structure which provides a seating surface readily retaining the ring in position during movement by the individual.

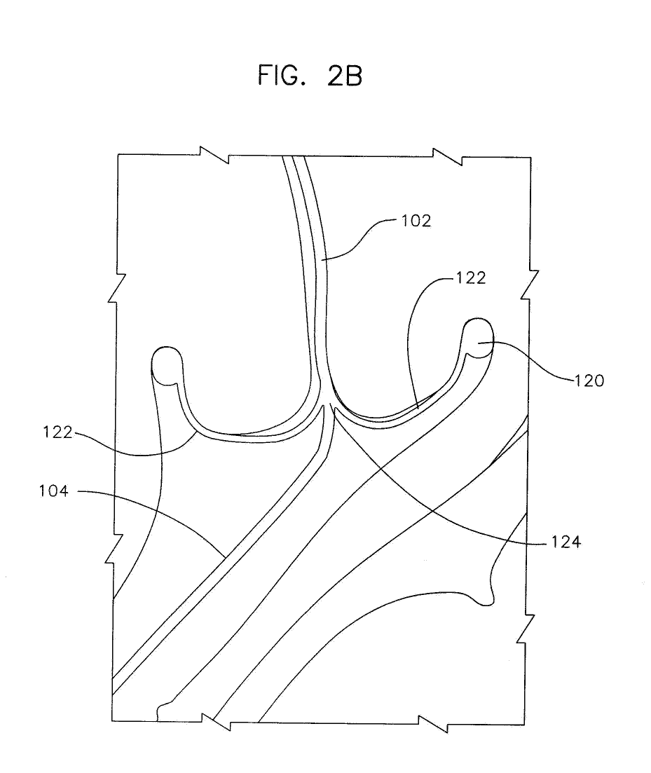

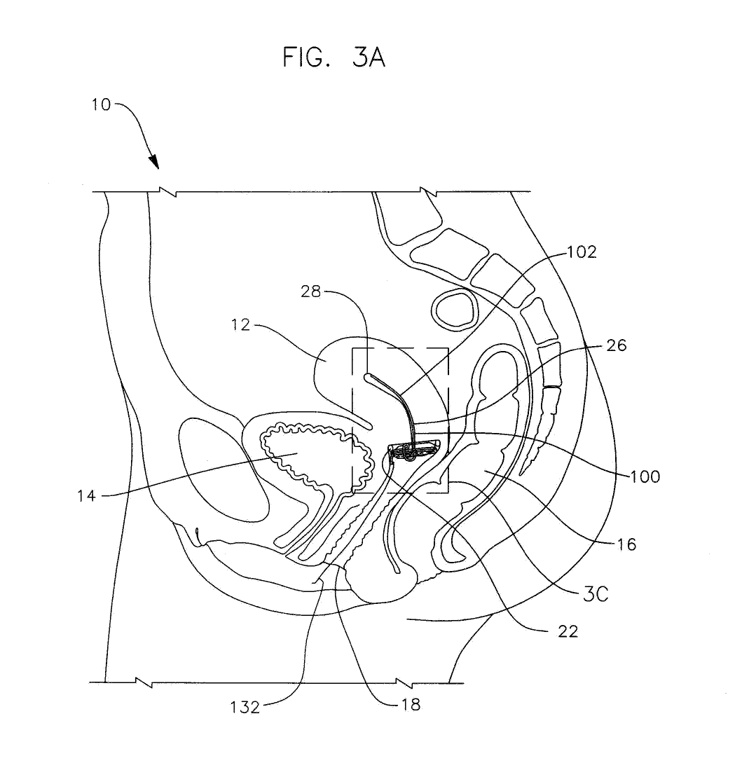

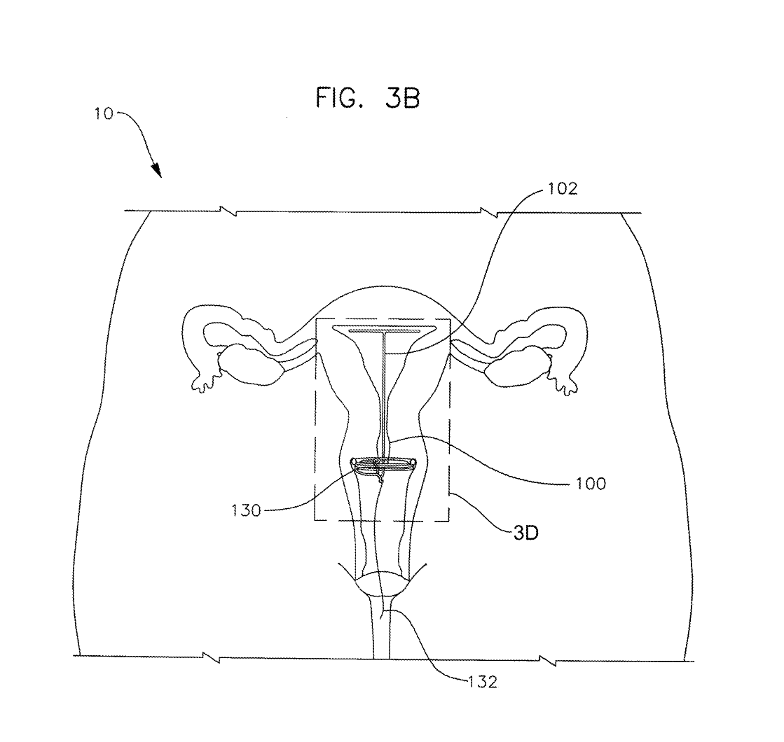

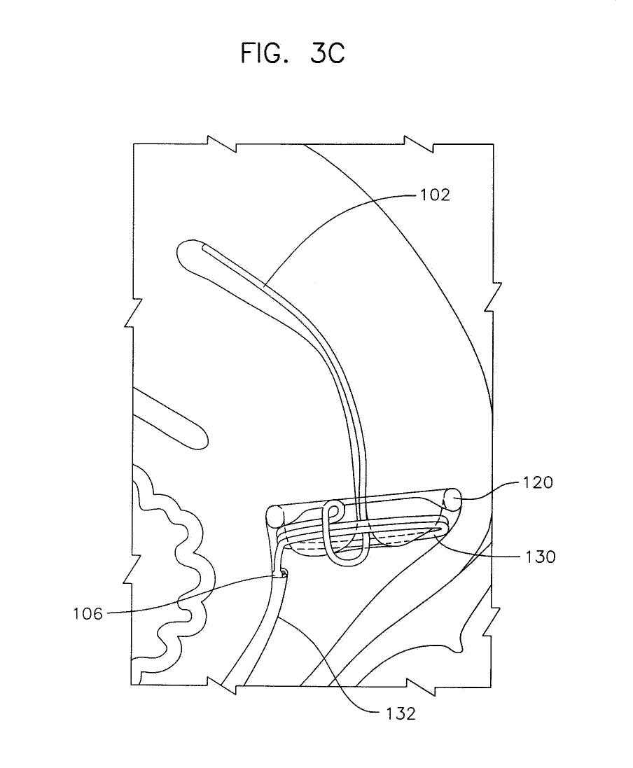

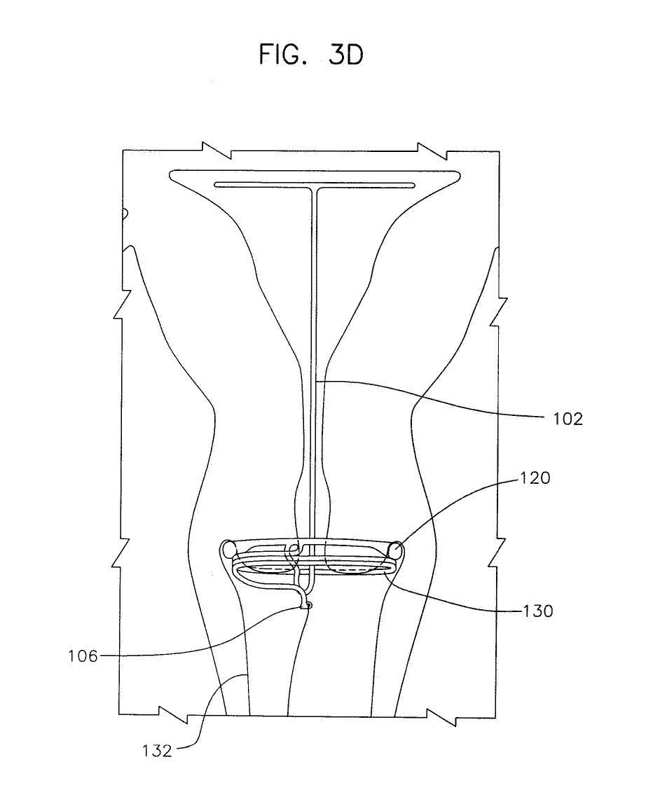

FIG. 3A is a schematic side cross sectional view of the pelvic region 10 depicting an alternative embodiment of a vasoconstrictor delivery system 100. As with prior embodiments, the pelvic region 10 of a woman is depicted, including the uterus 12 as positioned relative to the bladder 14 and colon 16. The uterus 12 is illustrated in a non-pregnant state as a generally collapsed structure located at the distal end of the vaginal canal 20. The vaginal opening 18 provides access to the uterus 12 through the vaginal canal 20. At the juncture of the vaginal canal 20 and uterus 12 is the fornix area 22 which forms a generally circular recess having the uterine cervix 24 protruding from within the area defined by the generally circular boundary of the fornix area 22. Within a central portion of the uterine cervix 24 lies the external ostium 26 which provides access to the cervical canal through the internal ostium and then the inside or interior 28 of the uterus.

As depicted in FIG. 3A, a vasoconstrictor delivery device 100 is inserted into the vaginal canal 20 through the vaginal opening 18. The delivery device 100 includes a catheter distal portion 102 extending into the uterus 12 or uterine cavity 28. Unlike prior embodiments, the proximal portion 130 of the delivery device 100 is a coiled catheter portion that is positioned within the fornix area 22. The proximal end of the coiled catheter portion does not extend outside the vaginal opening; rather a string 132 is affixed near the proximal end of the coil 130 and allows the individual to pull on the string to unfurl a portion of the coil 130. This action pulls the proximal end of the catheter out of the vaginal opening 18. Vasoconstrictor can then be injected into the delivery system through the proximal end after which the proximal portion or coil 130 can retract to its prior position within the fornix area 22. As with other embodiments, the proximal end 106 can include a septum or other type of isolatable connection for cooperation with an applicator or syringe when in use.

FIG. 3B is schematic frontal cross sectional view of the pelvic region 10 depicting the delivery system 100 of FIG. 3A having a coil section 130 positioned in the fornix area 22. In this exemplary embodiment, the distal portion 102 also includes a t-shaped distal end for distribution of the vasoconstrictor. As can be seen in more detail in FIG. 3C, the coil 130 can be a continuation of the distal portion 102 of the catheter which may be preformed into a coiled configuration that is sized to be retained within the fornix area 22. Further the proximal end is depicted with a septum for accessing the lumen of the catheter. As illustrated in FIG. 3D, the coil portion can be affixed to ring structure 120, like that of previous embodiments to prevent the entire coil from being pulled out when the string is pulled.

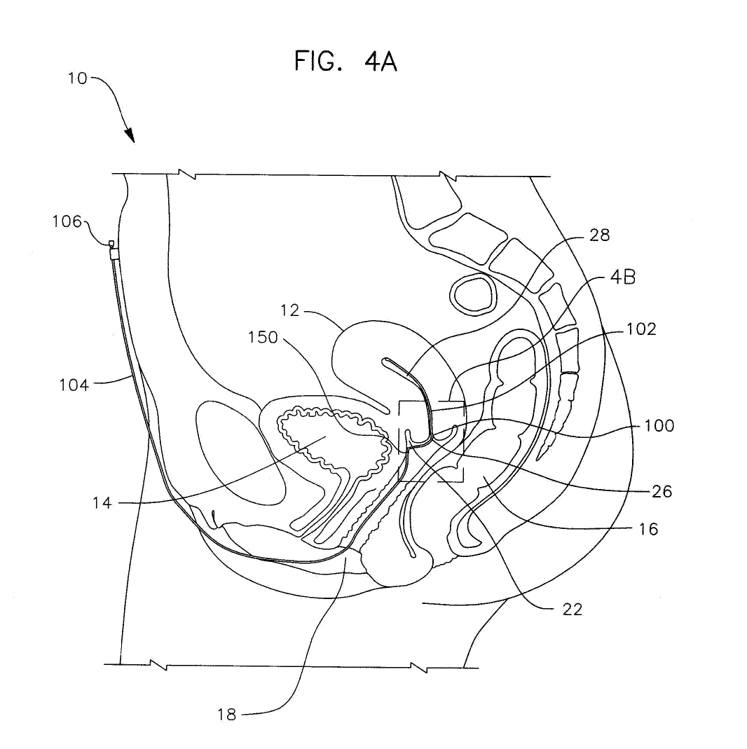

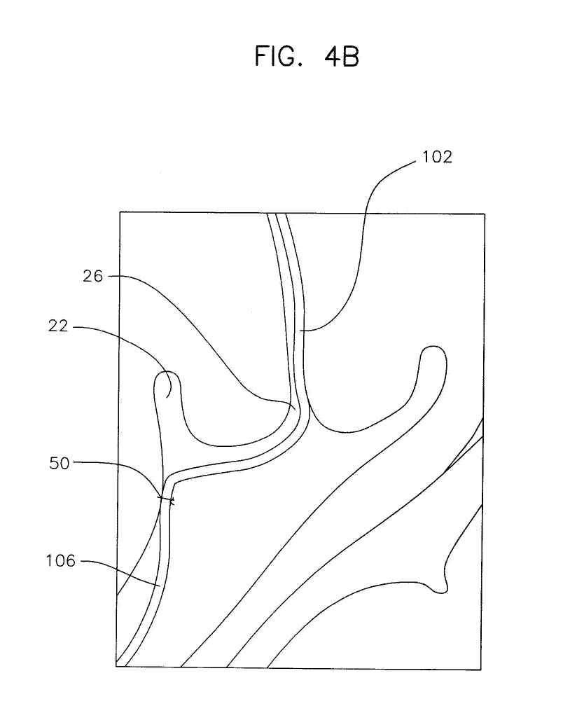

FIG. 4A is also a schematic side cross sectional view of the pelvic region 10 of a woman depicted to include the uterus 12 as positioned relative to the bladder 14 and colon 16. The uterus 12 is illustrated in a non-pregnant state as a generally collapsed structure located at the distal end of the vaginal canal 20. The vaginal opening 18 provides access to the uterus 12 through the vaginal canal 20. At the juncture of the vaginal canal 20 and uterus 12 is the fornix area 22 which forms a generally circular recess having the uterine cervix 24 protruding from within the area defined by the generally circular boundary of the fornix area 22. Within a central portion of the uterine cervix 24 lies the external ostium 26 which provides access to the cervical canal to the internal ostium and then the inside or interior 28 of the uterus.

As depicted in FIG. 4A, a vasoconstrictor delivery device 100 is inserted into the vaginal canal 20 through the vaginal opening 18. The delivery device 100 includes a catheter distal portion 102 extending into the uterus 12 or uterine cavity 28. A proximal portion 104 of the delivery device 100 extends a sufficient distance proximally to have a proximal end 106 accessible on the exterior surface of the pelvis for injection of a vasoconstrictor therein. The proximal end 106 can include a septum or other type of isolatable connection for cooperation with an applicator or syringe when in use. In the embodiment of FIG. 4A, a suture attachment 150 is included to affix the proximal portion of the catheter to the vaginal wall. Alternatively, the suture may attach the catheter to the cervix. The suture attachment 150 aids in retaining the distal portion 102 within the uterine cavity 28. FIG. 4B shows a more detailed view of the suture attachment 150 to the vaginal wall proximate the cervix.

FIG. 5A is a schematic side cross sectional view of the pelvic region 10 depicting an alternative embodiment of a vasoconstrictor delivery system 100 as positioned in the vaginal canal and uterine cavity. With this embodiment, a catheter proximal portion terminates proximally within the vaginal canal and includes a suture attachment to retain the position of the catheter. In some embodiments, the proximal end 106 of the catheter can include a docking port 154, as depicted in FIG. 5B, that mates with an applicator that is inserted into the vaginal canal for delivery of the vasoconstrictor.

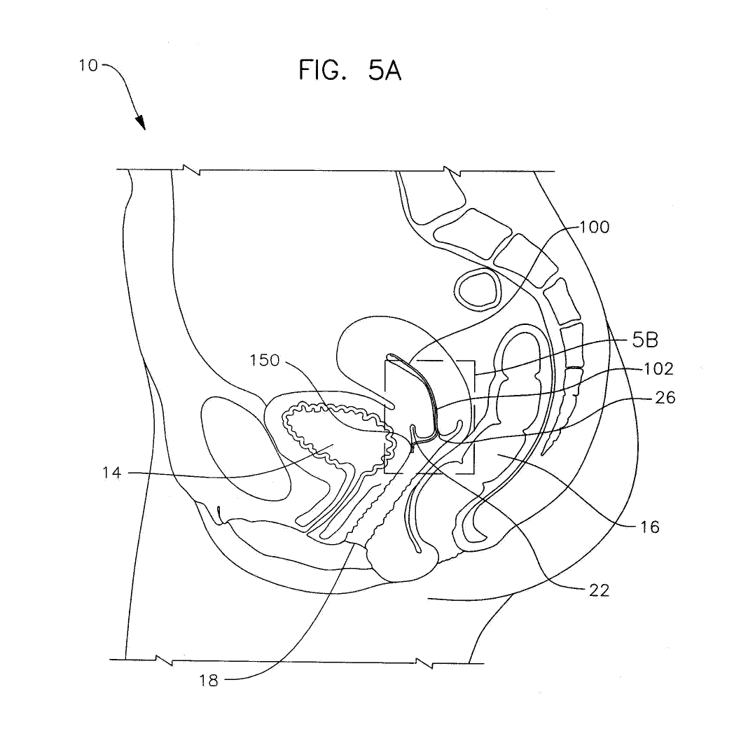

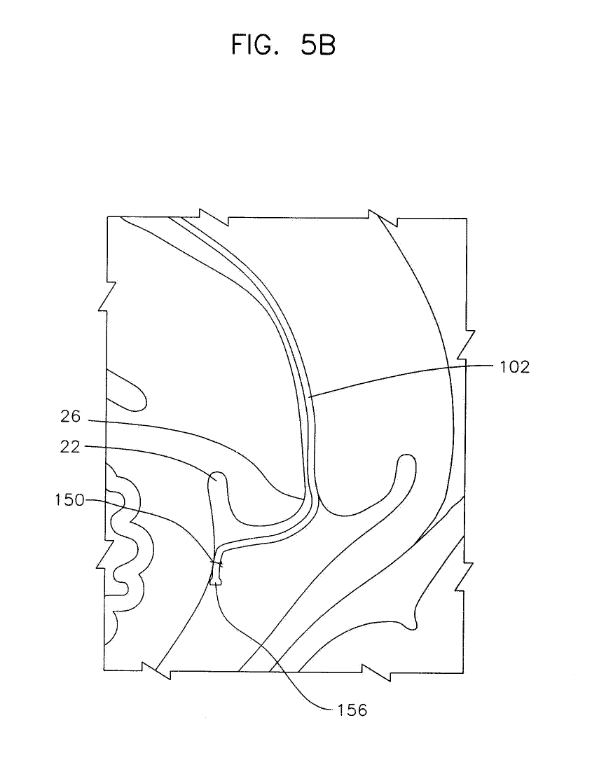

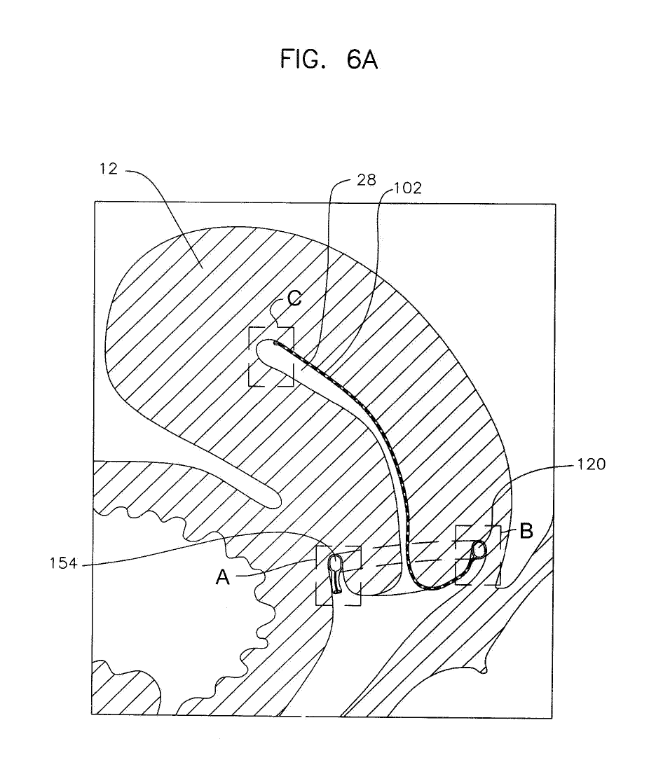

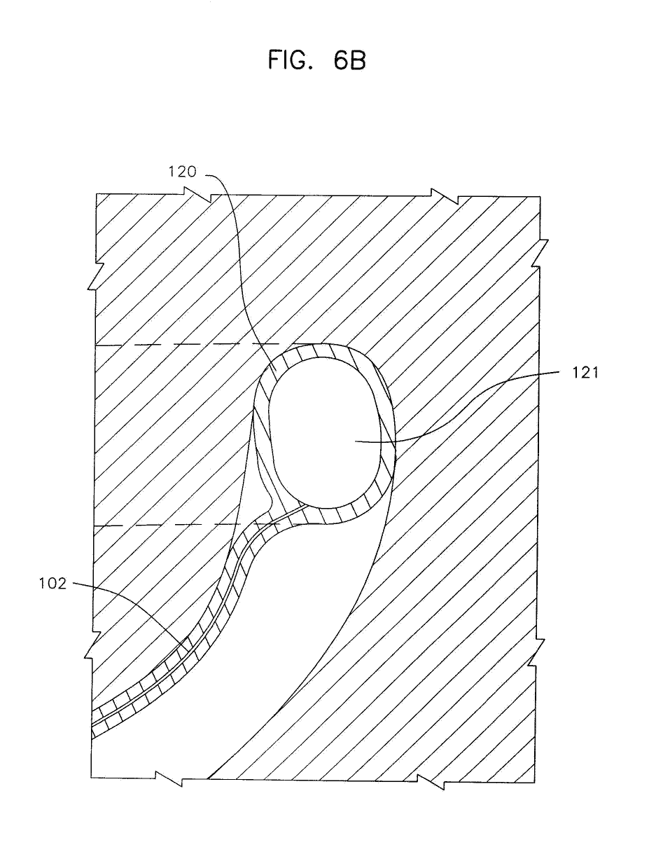

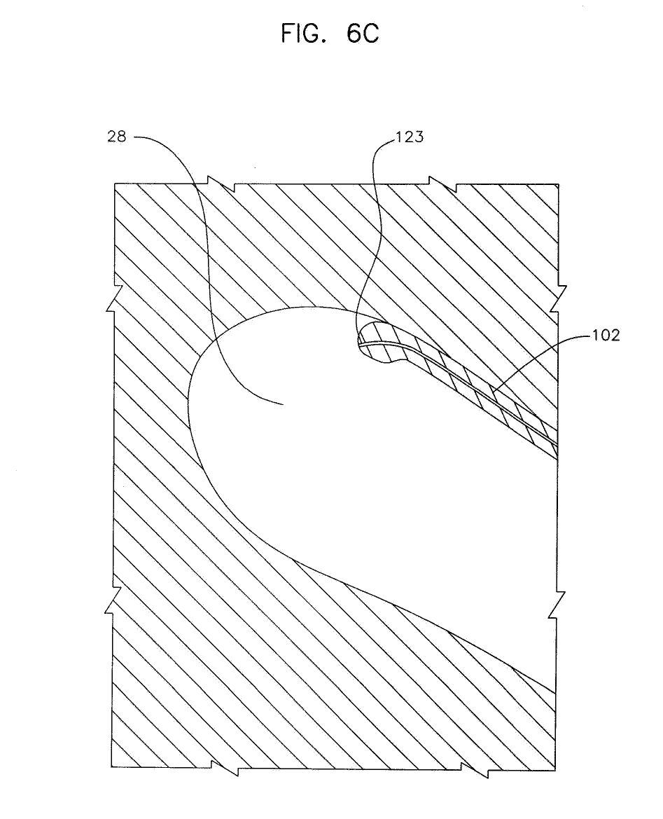

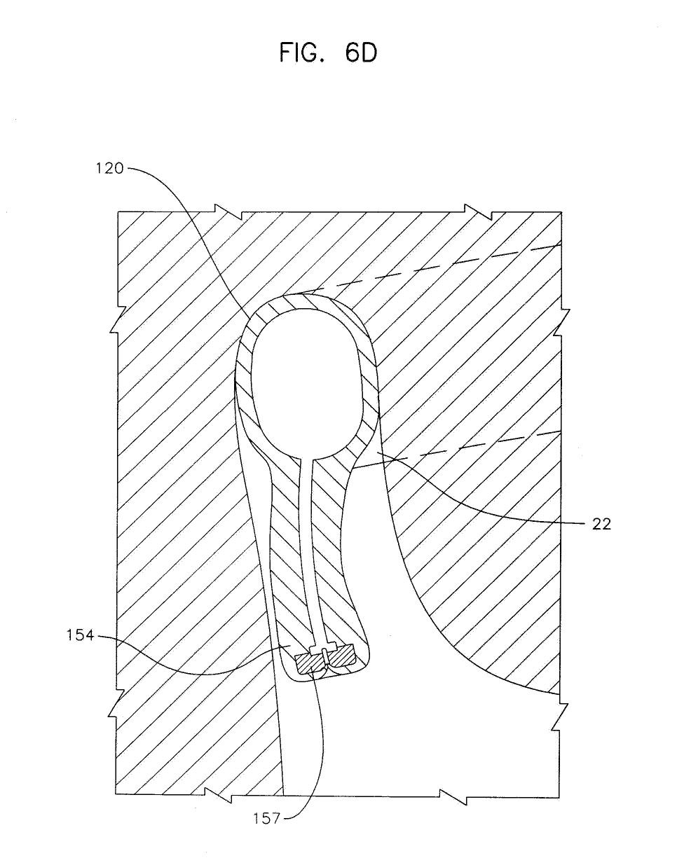

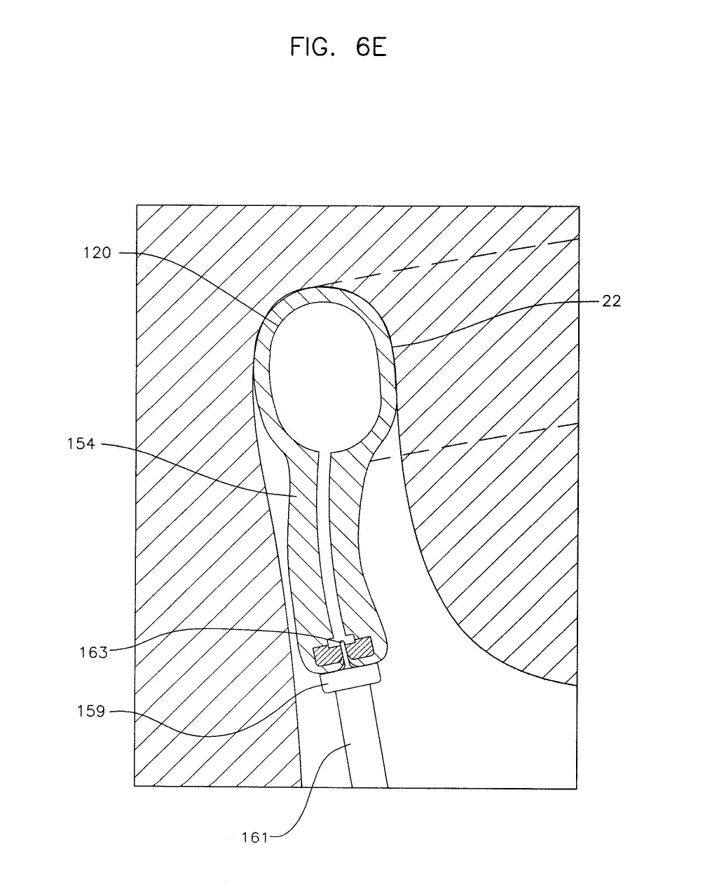

Other embodiments of a catheter having a proximal portion terminating in the vaginal canal can combine previous features disclosed. For example, FIG. 6A is partial cross sectional view of an alternative embodiment illustrating the combination of a ring structure 120 for retention of a distal catheter portion 102 and a docking port 154 located on the ring structure for coupling to a source of vasoconstrictor. The ring structure 120 can be in the form of a hollow reservoir 121 as depicted in FIG. 6B. The ring structure 120 could include a pump for dispensing a therapeutic amount of vasoconstrictor as needed. The vasoconstrictor would exit a distal port 123 within the uterine cavity 28 as depicted in FIG. 6C. Coupling to the docking port would be necessary to replenish the reservoir. Alternatively, the ring structure 120 can be present to position and hold the docking port 154 in a preferred location for docking with a source of vasoconstrictor. In this embodiment, each time a dose of vasoconstrictor is needed, an applicator will be inserted into the vaginal canal to couple with the pre-positioned docking port. A representative docking port 154 is depicted in more detail in FIG. 6D. The docking port can include a septum 157 that is penetrable by an applicator sized to couple therewith. A distal portion 159 of an applicator 161, as docked with the port is depicted in FIG. 6E. A distal projection 163 penetrates the septum and allows fluid communication with the lumen of the vasoconstrictor delivery system 100.

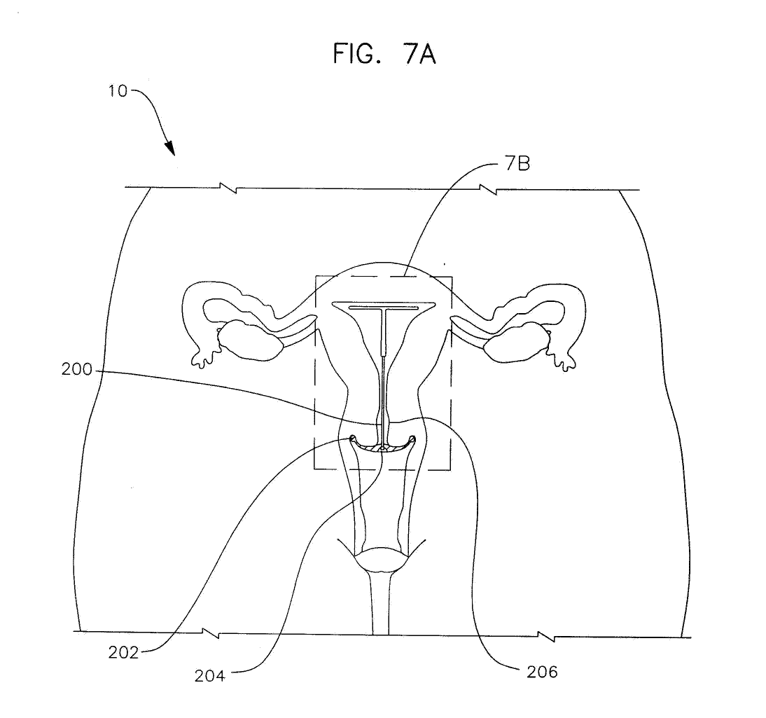

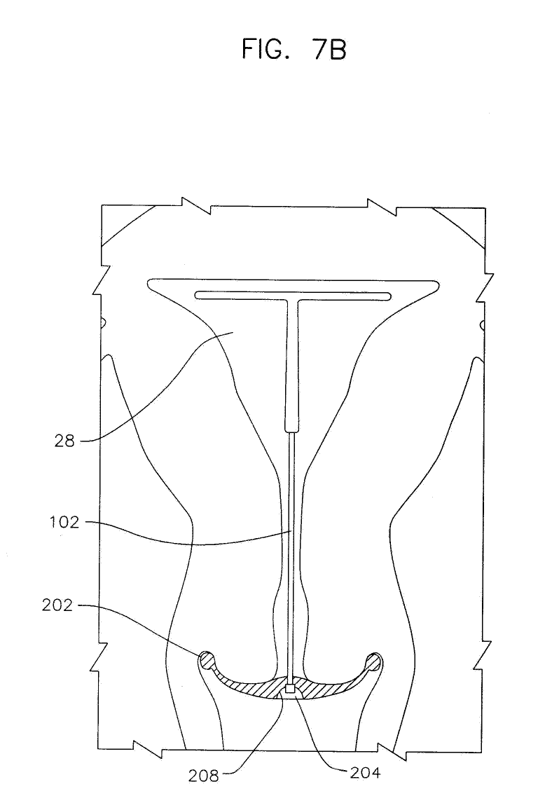

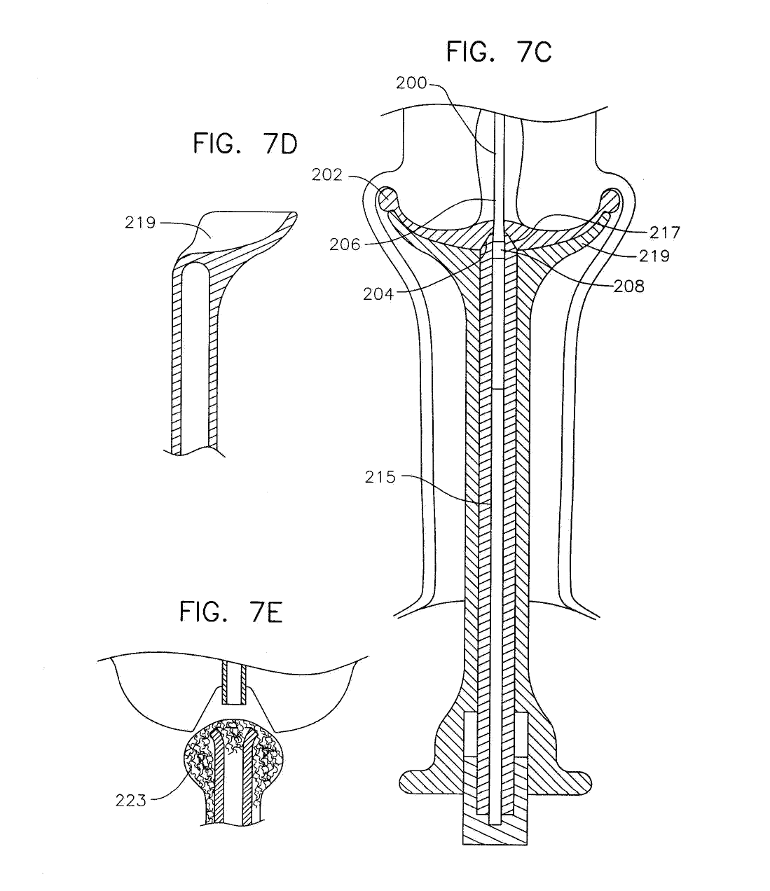

FIG. 7A is a schematic frontal cross sectional view of the pelvic region 10 depicting an alternative delivery system 200 having a retention ring 202 and a docking port 204 located in alignment with the cervical canal 206. As depicted in more detail in FIG. 7B, the centrally located docking port 204 can include a funnel shaped receptacle 208 for receiving a distal portion of an applicator device. The funnel shaped receptacle 208 can be in fluid communication with the distal portion of the catheter 102 that extends into the uterine cavity 28.

FIG. 7C depicts one alternative applicator device 215 schematically as docked with the port 204 of FIG. 7B. A cone shaped distal end 217 of the applicator fits within the funnel shaped receptacle 208 to form a fluid tight seal that allows injection into the lumen of the catheter 102. A septum can also be included that is penetrated by the distal end of the applicator.

The applicator can also include structure to aid in positioning the distal end in the funnel shaped receptacle. As illustrated in FIG. 7C and in more detail in FIG. 7D, the distal portion of the applicator can include a partial cup-like portion 219 that contacts the fornix area 22 or the ring 202 for positioning the applicator in relation to the docking port 204.

In another alternative feature of the applicator of FIG. 7C, the distal portion of the applicator that fits within the funnel shaped receptacle can include means for cleansing the port 223 prior to injection of vasoconstrictor. For example, as depicted in FIG. 7E, a fibrous distal end 223 can include a cleanser that is wiped onto the port as the applicator is introduced into the funnel-shaped receptacle. With this design, before the applicator seals with the port, the port has been wiped clean.

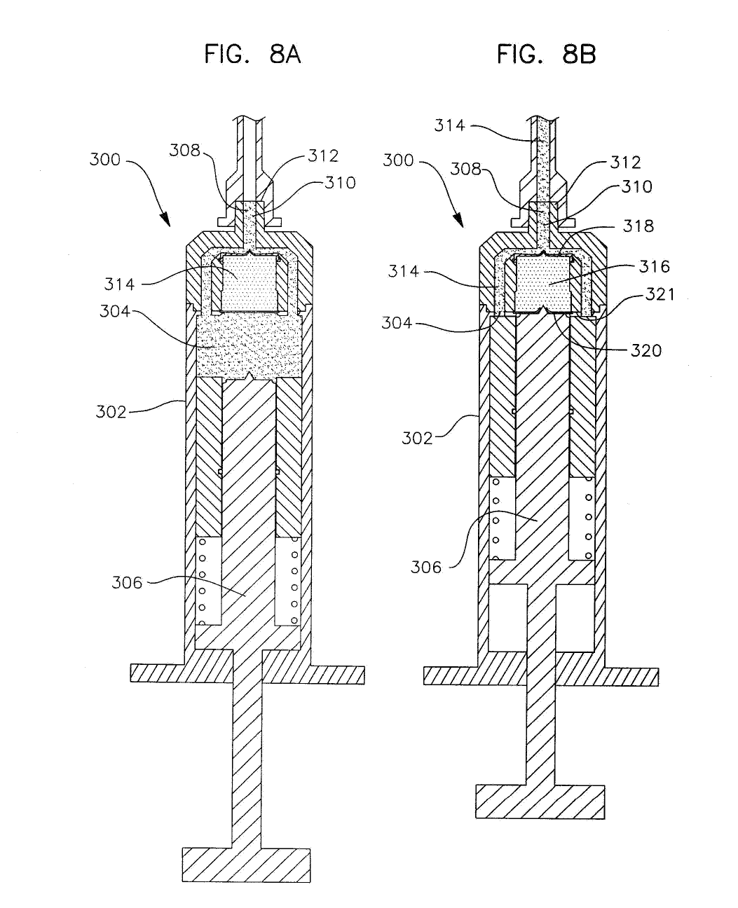

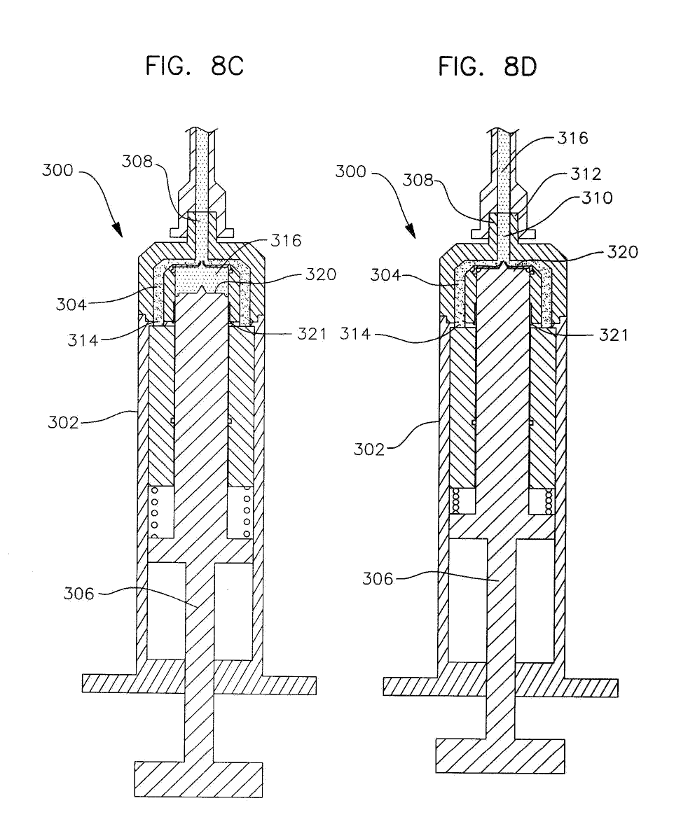

FIGS. 8A-8D are schematic cross sectional views of applicators 300 that can interface with at least some of the docking ports of the various embodiments of delivery systems disclosed. In particular, the illustrated applicators 300 include provision for dispensing a cleansing solution prior to injection of the vasoconstrictor.

As seen in FIG. 8A, an exemplary applicator 300 can include a syringe having a chamber 304 with a plunger 306 slidably received therein. The distal end of the syringe 302 includes a discharge port 308 having a lumen 310 in fluid communication with the chamber 304. The discharge port 308 can include a coupling 312 that cooperates with any of the various embodiments of vasoconstrictor delivery systems disclosed herein. As shown in FIG. 8A, the coupling can include a luer type fitting wherein the discharge port includes a nozzle that fits into a lumen of the delivery system port and seals therewith for injection of vasoconstrictor.

Also as shown in FIG. 8A, the syringe can include multiple internal chambers 314, 316 carrying different therapeutic materials that are injected in stages as the plunger 306 is moved distally. In FIG. 8A, the syringe includes a first chamber 314 having a disinfectant and a second chamber 316 containing the vasoconstrictor, with each chamber isolated from the other. As indicated in FIG. 8B, initial distal movement of the plunger 306 forces the disinfectant out the discharge port 308 around the outside of the second chamber 316. The second chamber 316 is a smaller diameter and includes a distal seal 318 that ruptures if a set pressure is reached. The entire plunger moves distally to contact the second chamber wherein walls 321 of the second chamber act as a stop for the outer portion of the plunger while an internal portion continues to move distally by forcing a proximal wall 320 of the second chamber in a distal direction. This movement is illustrated in FIG. 8C. The vasoconstrictor is ejected until the plunger seats at the distal end of the chamber as indicated in FIG. 8D.

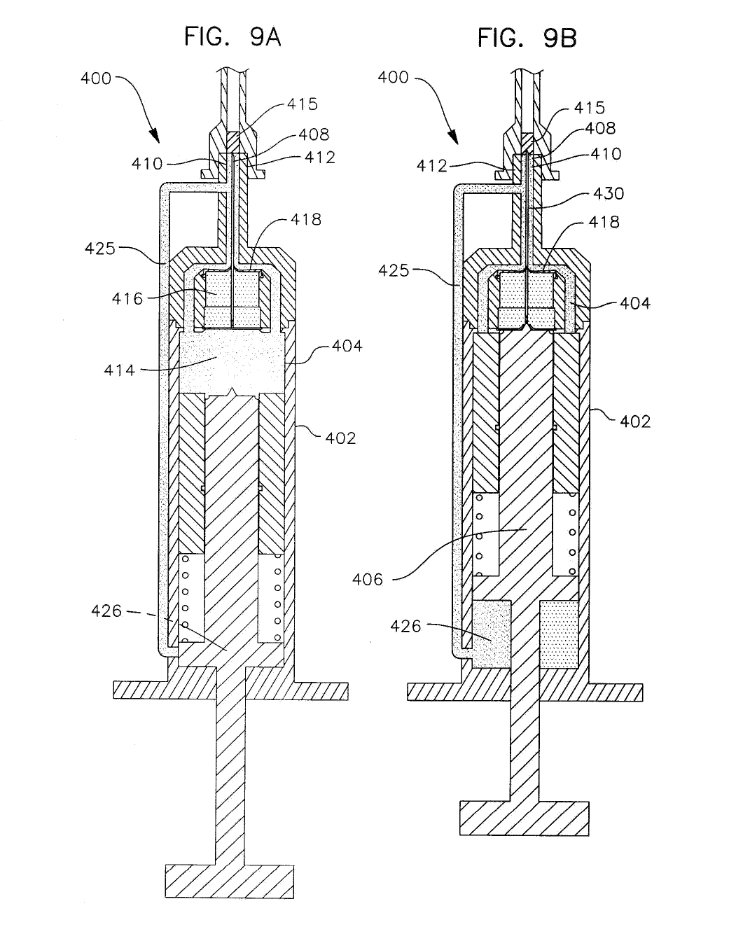

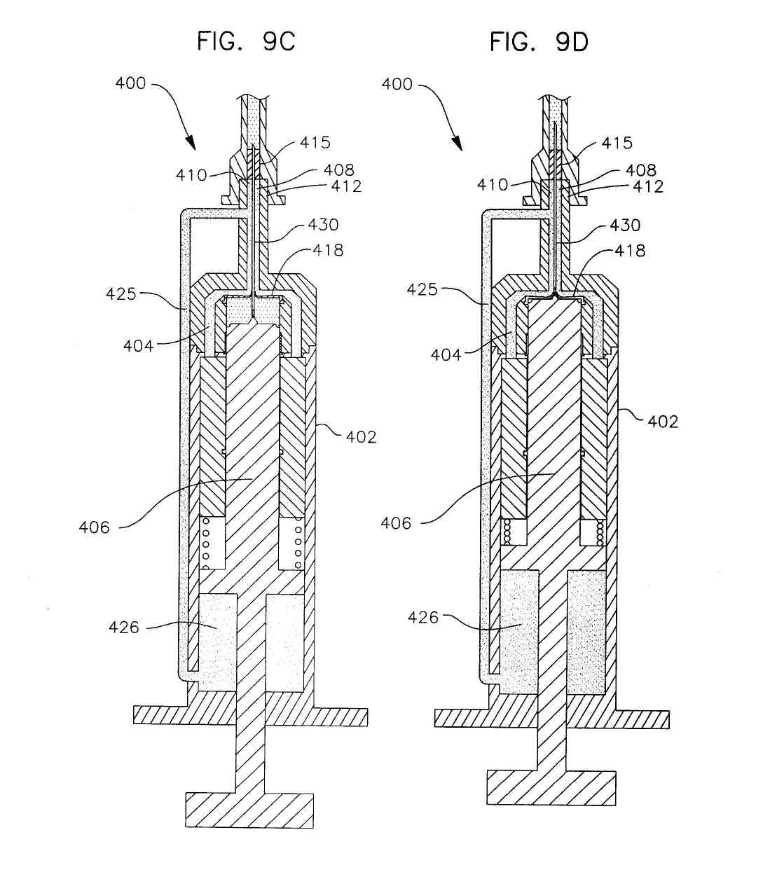

FIGS. 9A-9D are schematic cross sectional views of an alternative applicator system 400 that includes recycling of a cleansing fluid that cleans a septum area of the delivery system port prior to injection of the vasoconstrictor. The design of the applicator is a syringe 402, similar to that of FIG. 8A. The distal end of the syringe includes a discharge port 408 having a lumen 410 in fluid communication with at least one chamber 404. The discharge port can include a coupling that cooperates with any of the various embodiments of vasoconstrictor delivery systems disclosed herein. As shown in FIG. 9A, the coupling can include a luer type fitting wherein the discharge port 408 includes a nozzle 412 that fits into a lumen of the delivery system port and seals therewith for injection of vasoconstrictor. With the embodiment of FIG. 9A, the delivery system port includes a septum 415 that can be pierced or has a sealing slit therethrough. As indicated in FIG. 9A, the syringe mates with the delivery system port and first engages, but does not pierce or couple in fluid communication with the lumen beyond the septum. In this first position, the disinfectant or cleansing material is ejected to clean the septum area. The syringe of FIG. 9A includes a recycle tube 425 having a lumen extending to the proximal side 426 of the plunger 406 and opens into the chamber on that side of the plunger. As disinfectant is discharged, as show in FIG. 9B, the cleansing agent washes over the septum area and is returned to the syringe proximal of plunger.

Also as with the embodiment of FIG. 8A and shown in FIG. 9A, the syringe can include multiple internal chambers 414, 416 carrying different therapeutic materials that are injected in stages as the plunger is moved distally. In FIG. 9A, the syringe includes a first chamber 414 having a disinfectant and second chamber 416 containing the vasoconstrictor, with each chamber isolated from the other. As indicated in FIG. 9B, initial distal movement of the plunger 406 forces the disinfectant out the discharge port 408 around the outside of the second chamber 416. The second chamber 416 is a smaller diameter and includes a distal seal 418 that ruptures if a set pressure is reached. With the embodiment of FIG. 9B, an extendable needle 430 resides in the second chamber 416 and extends distally to near the distal end of the syringe. The entire plunger moves distally to contact the second chamber wherein walls of the second chamber act as a stop for the outer portion of the plunger while an internal portion continues to move distally by forcing a proximal wall of the second chamber in a distal direction. This movement is illustrated in FIG. 9C and first causes the needle 430 to move distally and pierce the septum 415 to be in fluid communication with the lumen of the delivery system. Vasoconstrictor fills the lumen of needle via a port near the proximal end of the needle. The vasoconstrictor is ejected until the plunger seats at the distal end of the chamber as indicated in FIG. 9D.

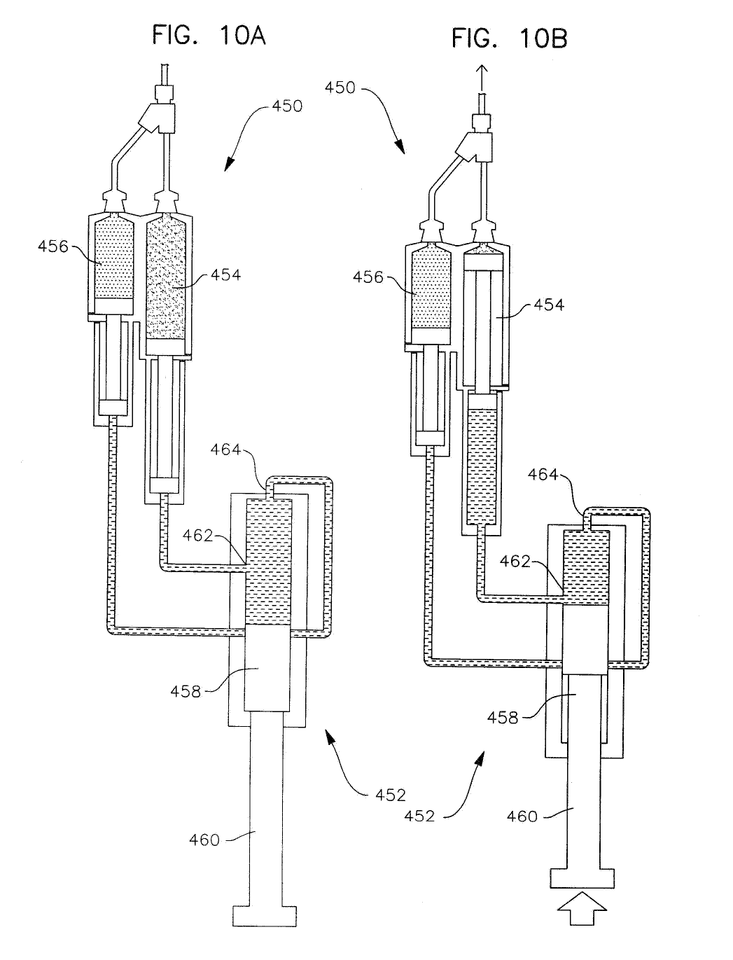

FIGS. 10A-10D are schematic cross sectional views illustrating a multi-reservoir applicator 450 that can allow for staged injection of various media by movement of a single plunger. The applicator is shown schematically for better understanding of the operation of the parts recognizing that a final assembly would place the components in a compact structure and relationship. In general, the applicator 450 includes a hydraulic valve 452 that is connected to multiple chambers. For example, as indicated in FIG. 10A, the hydraulic valve 452 is connected to a first chamber or first syringe 454 having a disinfectant therein. The hydraulic valve 452 is also connected to a second chamber or second syringe 456 having the vasoconstrictor therein. The hydraulic valve 452 is itself a syringe 458 having a plunger 460 and a proximal discharge port 462 and a distal discharge port 464.

As seen in FIG. 10A, in a first position with the hydraulic valve plunger 460 retracted proximally, the proximal port 462 is in fluid communication with the hydraulic valve chamber while the distal port 464 is blocked by the plunger as it includes a tube that passes back through the chamber on a proximal portion of the hydraulic valve chamber that is blocked by the plunger 460. As indicated in FIG. 10B, initial distal movement of the hydraulic valve plunger 460 causes discharge through the proximal port that forces a plunger on the disinfectant syringe distally to eject the disinfectant.

When the plunger 460 blocks the proximal port 462 as it moves distally, as indicated in FIG. 10C, flow of disinfectant stops and a flow path opens from the distal port 464 of the hydraulic valve 452 through the tube that goes around the back side of the plunger 460 and into the syringe 456 having the vasoconstrictor therein. As indicated in FIG. 10D, continued distal movement of the hydraulic valve plunger 460 cause distal movement of the plunger on the vasoconstrictor syringe 456 to eject the vasoconstrictor. One advantage of the hydraulic valve 452 is the elimination of the need for an isolated second chamber as was included in the previous embodiments. The isolated chamber required a membrane that ruptures to release the fluid.

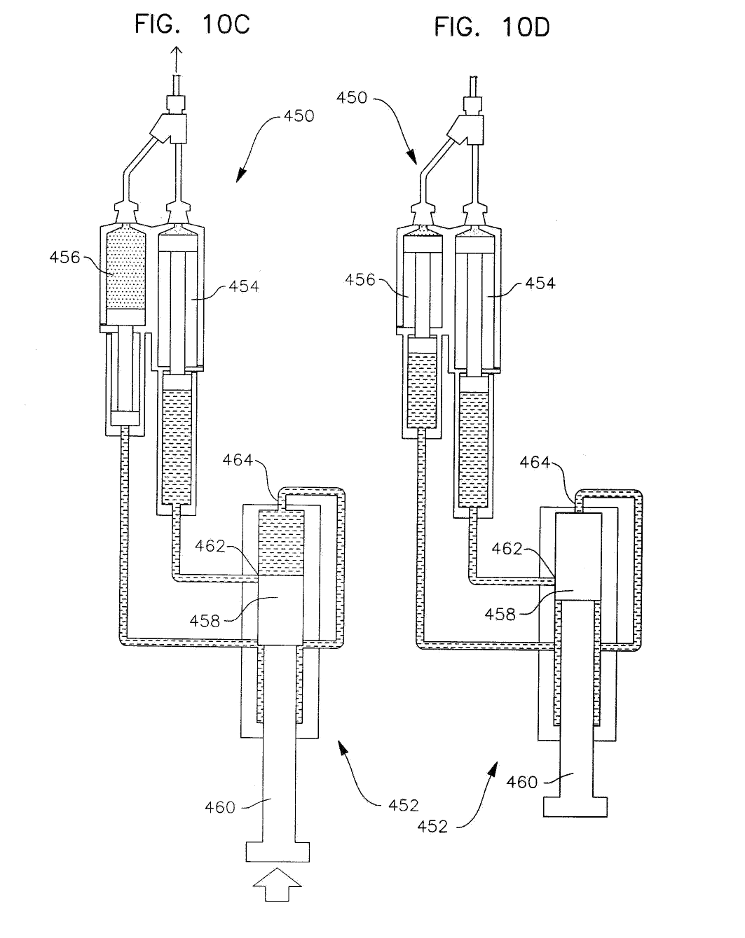

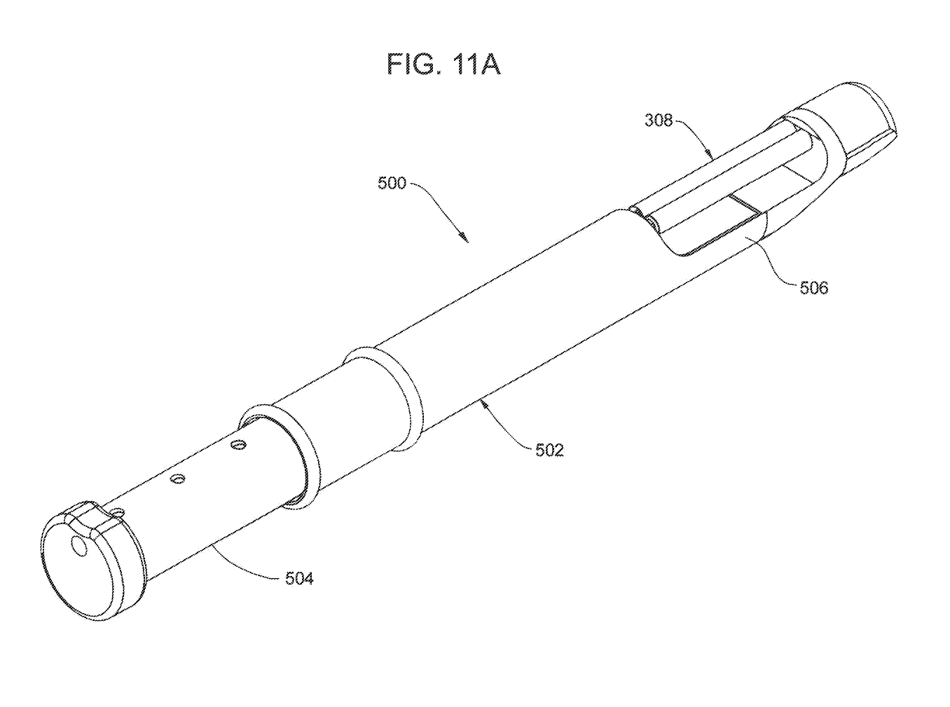

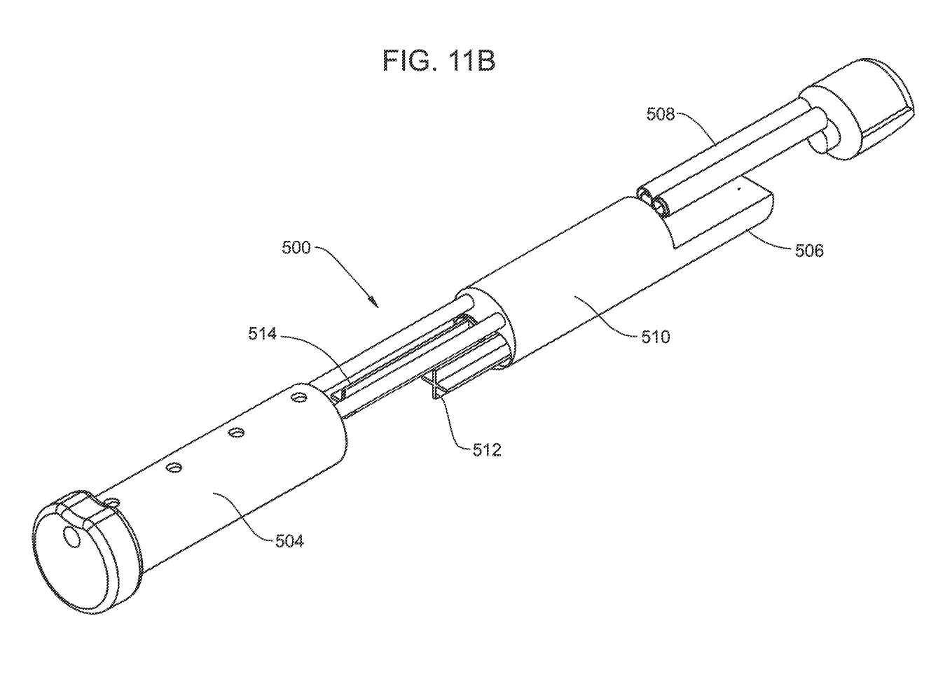

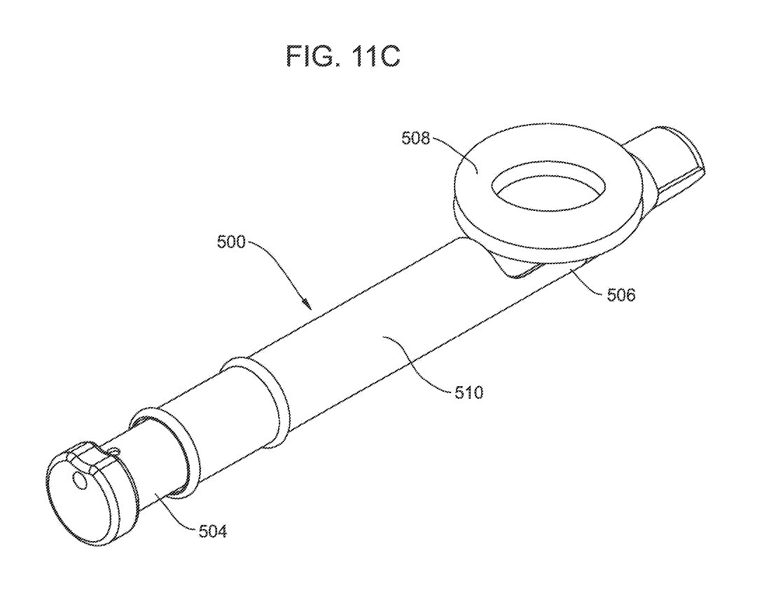

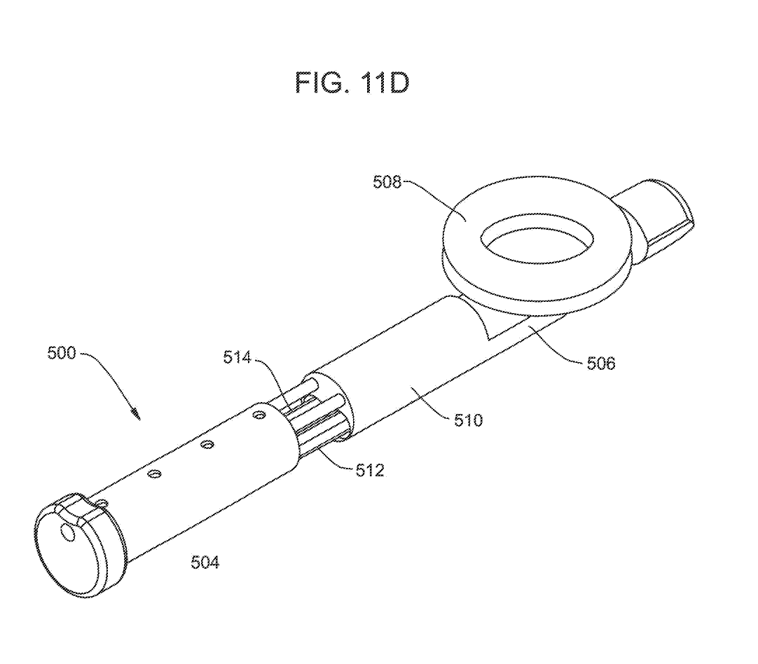

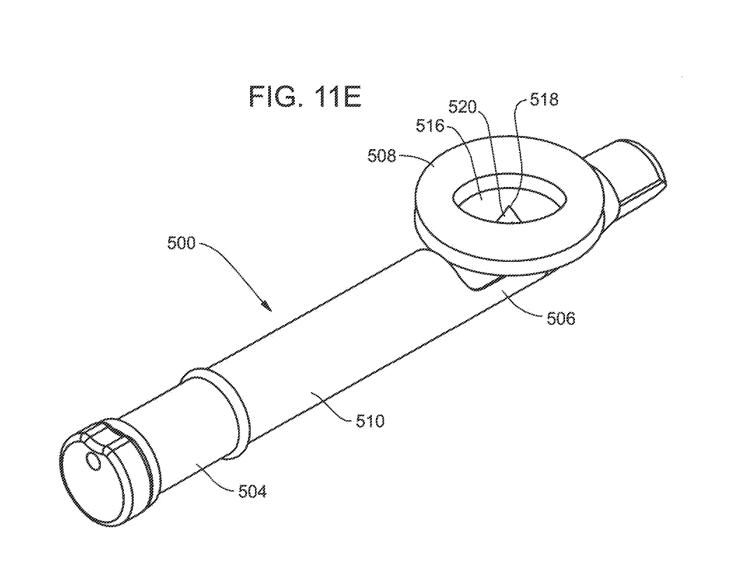

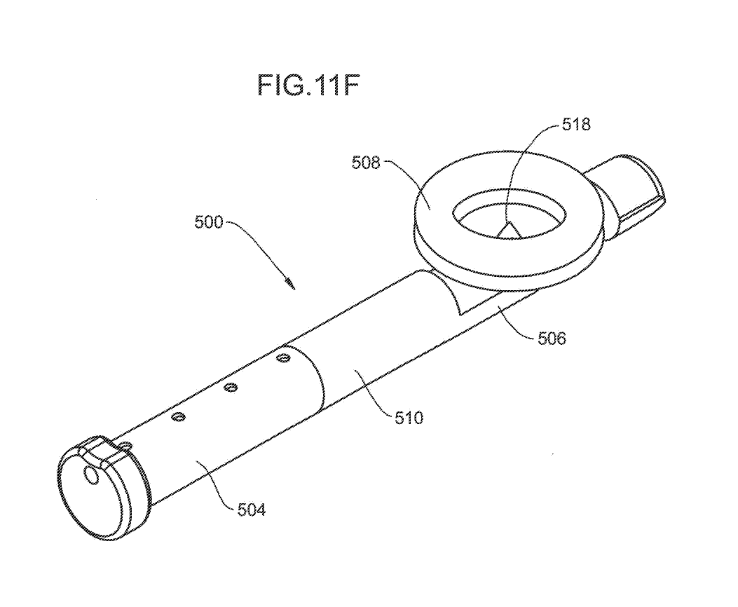

FIGS. 11A-11F are schematic perspective views of an alternative vasoconstrictor applicator in various stages of deployment. The vasoconstrictor applicator is designed to mate directly with the cervical canal or the outer ostium of the cervical canal. With this embodiment, a catheter having a distal portion inside of the uterus is not necessary. Further, the applicator does not require a docking port positioned within the vaginal canal as was disclosed with respect to prior embodiments. All components are included with the applicator and upon delivery of the vasoconstrictor are removed from the vaginal canal. Nothing, except the vasoconstrictor, is left in the vaginal canal or in the uterus between doses with this applicator and delivery system combination.

Referring to FIG. 11A, a cervical positioning applicator assembly 500 for delivering a topical vasoconstrictor into the uterus is depicted in a pre-deployment configuration. The applicator assembly 500 includes a shaft 502 having a distal portion 506 including an expandable positioning ring 508. The expandable positioning ring 508 is depicted in a first unexpanded profile for insertion in the vaginal canal.

The shaft 502 can include a reservoir to carry the vasoconstrictor within a reservoir housing 510 or multiple reservoirs within the reservoir housing 510, as better illustrated in FIG. 11B, which is a schematic perspective view of the applicator of FIG. 11A with the outer sheath removed. Additional reservoirs can include inflation fluid for deployment of the applicator embodiments having inflatable membranes included in the distal portion of the applicator. Alternatively, the reservoir housing 510 can include mechanisms for deployment of the applicator in embodiments that include a self expanding distal portion that must be released to deploy.

FIG. 11B also schematically illustrates a plunger assembly 504 and elements of a mechanism for staged deployment of the applicator with distal movement of the plunger assembly 504. The plunger assembly 504 can include a first push rod 512 in fluid communication with a first reservoir and a second push rod 514 in fluid communication with a second reservoir. As depicted the plunger assembly is pushed distally with manual force by the user. Alternatively, the plunger assembly can include a spring that stores the necessary energy to deploy the device and inject the vasoconstrictor. The spring would be stretched or cocked for actuation and when released, pull the plunger assembly distally.

Now referring to FIG. 11C, the expandable positioning ring 508 is depicted after deployment in a second expanded profile. In use, the applicator is inserted into the vaginal canal prior to deployment of the expandable positioning ring 508. Upon deployment, the expandable positioning ring is placed generally in contact with the fornix area in the upper vaginal canal. In this position, the second expanded profile, a central axis of the ring is generally over the uterine cervix, more particularly over the outer ostium of the cervical canal that leads to the inside of the uterus.

As illustrated in FIG. 11D, wherein the outer sheath of the shaft has been removed, it can be seen that the plunger actuator has been moved distally to move the first push rod 514 which in turn has deployed the expandable positioning ring 508. In alternative embodiments, the positioning ring can be an inflatable membrane and distal movement of the plunger actuator and the first push rod 514 cause inflation fluid to fill and expand the positioning ring 508. In another alternative embodiment, the distal portion of the shaft can include a retractable sheath disposed about at least the distal portion of the shaft. In this embodiment the positioning ring can include a self-expanding o-ring retained in an unexpanded profile by the sheath and deployed to an expanded profile by retraction of the sheath. Initial movement of the plunger actuator causes the sheath to rotate or move proximally to release the positioning ring 508.

Now referring to FIG. 11E, the applicator is illustrated with an injection assembly 516 deployed. The injection assembly 516 can include a distal projection 518 located in the central portion of the positioning ring 508 and an injection port 520 thereon. In some embodiments, the injection assembly 516 having the injection port 520 thereon can be positioned within the circumference of the positioning ring. The injection assembly 516 can include an inflatable membrane having a first retracted position and upon inflation of the membrane moves to a second inflated position with the injection port 518 moving along the central axis of the positioning ring 508 into generally sealing contact with the cervix. The distal projection is sized to fluidly couple with the lumen of the cervix for injection of a vasoconstrictor therein. Final distal movement of the plunger actuator causes the injection of vasoconstrictor through the injection port 518 into the uterus.

In some alternative embodiments, the shaft can include multiple reservoirs and a plunger assembly, wherein a first reservoir includes an inflation fluid for expanding the positioning ring and a second reservoir includes a second inflation fluid for expanding the injection assembly and a third reservoir contains a vasoconstrictor. In this embodiment, the plunger assembly can include a first push rod in fluid communication with the first reservoir, a second push rod in fluid communication with the second reservoir, and a third push rod in fluid communication with the third reservoir. The plunger actuator is slidably disposed within the shaft, wherein distal movement of the actuator first contacts the first push rod to force fluid to deploy the positioning ring and then contacts the second push rod to force fluid to expand the injection assembly and lastly contacts the third push rod to eject the vasoconstrictor. In other embodiments including a self-expanding positioning ring, the shaft can include a sheath that retracts to deploy the self-expanding positioning ring which may be in addition to or replace one of the reservoirs.

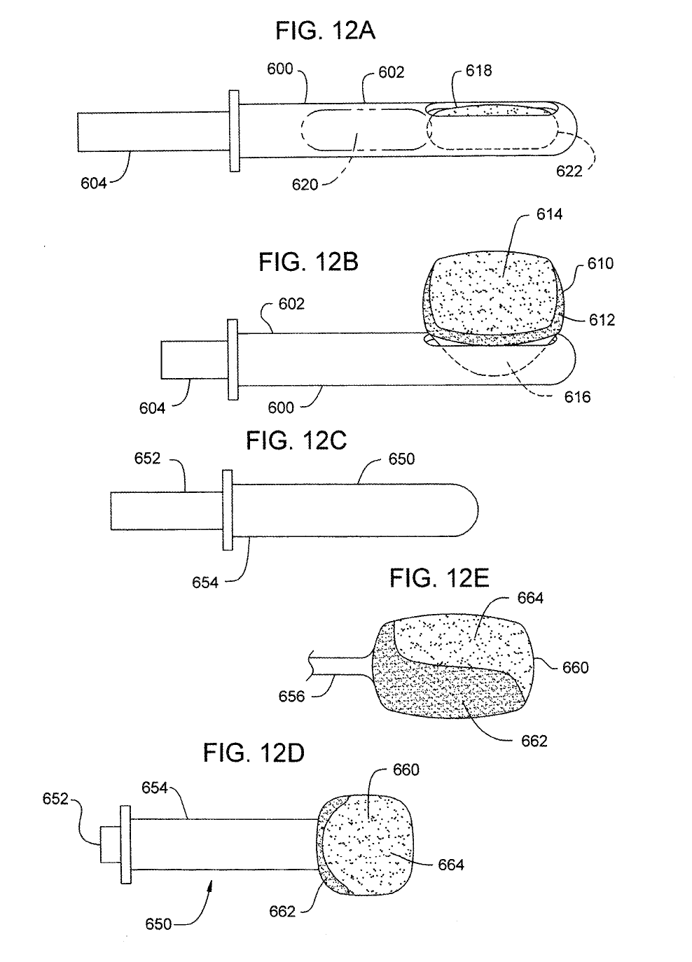

Now referring to FIGS. 12A-12E, schematic perspective views of an alternative vasoconstrictor applicator in various stages of deployment are depicted. The vasoconstrictor applicator is similar to that disclosed with respect to FIG. 11 in that it is designed to mate directly with the cervical canal or the outer ostium of the cervical canal. With this embodiment, a catheter having a distal portion inside of the uterus is not necessary. Further, the applicator does not require a docking port positioned within the vaginal canal as was disclosed with respect to prior embodiments. All components are included with the applicator and upon delivery of the vasoconstrictor are removed from the vaginal canal. Nothing, except the vasoconstrictor, is left in the vaginal canal or in the uterus between doses with this applicator and delivery system combination. In contrast to the embodiment of FIG. 11 the distal portion of the shaft carries an expandable foam member and some preferred embodiments include a means for compressing the foam member to force vasoconstrictor therefrom into the cervical canal when the foam member abuts at least a portion of the cervix.

Referring to FIG. 12A, a cervical positioning applicator assembly 600 for delivering a topical vasoconstrictor into the uterus is depicted in a pre-deployment configuration. The applicator assembly 600 includes a shaft 604 having a distal portion including an expandable foam member 620 (shown compressed and in phantom). The shaft 604 and expandable foam member 620 are slidably received within a sheath portion 602 of the shaft 604. The sheath portion includes a window 618 positioned on longitudinal surface with size and position configured so that upon insertion of the applicator into the vaginal canal, the window 618 is positioned over at least a portion of the cervix. The expandable foam member 620 can be saturated with vasoconstrictor within the sheath 602. Alternatively or additionally the shaft 604 can include a reservoir containing vasoconstrictor that may be added to the foam member 620 upon or during expansion. For example, the sheath 602 in which the foam member 620 resides can include additional vasoconstrictor that is absorbed into the foam member 620 as it expands to remain generally saturated with the vasoconstrictor in the expanded state.

Now referring to FIG. 12B, the applicator of FIG. 12A is depicted in a deployed or expanded state. As is illustrated in this embodiment, the shaft 604 has been moved distally so that the foam member 620 has aligned with the window 618 and expanded therethrough. When this is done within the vaginal canal, the upper surface 610 of the foam member 620 is placed in contact with at least a portion of the cervix and may also be in contact with the fornix area. As also depicted in FIG. 12B, the foam member 620 can include a central portion 614 made from an absorbent open cell foam that carries the vasoconstrictor and a perimeter portion 612 made from a closed cell foam that is generally impervious to the vasoconstrictor and can provide both a barrier and seal that is positioned to direct vasoconstrictor up into the cervical opening during use. When the foam member 620 abuts the cervix, a compressive force is needed to squeeze or compress the foam member to force the vasoconstrictor out of the foam into the cervical canal and into the uterus. In the embodiment of FIG. 12B, the foam member 620 is mounted to a balloon member 616 that upon inflation presses the foam member toward the cervix. This compressive force acts in the same way as squeezing a saturated sponge and with the closed cell foam directing the flow, the vasoconstrictor is forced into the cervical canal and into the uterus. The shaft 604 can include a reservoir to carry the vasoconstrictor within a reservoir housing or multiple reservoirs within the reservoir housing. Additional reservoirs can include inflation fluid for deployment of the applicator embodiments having inflatable membranes included in the distal portion of the applicator.

Now referring to FIG. 12C, an alternative embodiment of the applicator 650, similar to that of FIG. 12A is depicted. This embodiment includes a shaft 652 slidably disposed within a sheath 654. An expandable foam member 660 is mounted to a distal portion of the shaft 652 and resides in a compressed state within the sheath 654. The expandable foam member 660 is depicted in a deployed state in FIG. 12D. As illustrated, the shaft 652 is moved distally so that the foam member 660 extends beyond the distal end of the sheath 654 and the foam member can assume its uncompressed or expanded state. When this is done within the vaginal canal, the upper surface of the foam member 660 is placed in contact with at least a portion of the cervix and may also be in contact with the fornix area. As also depicted in FIG. 12D, the foam member 660 can include a central portion 664 made from an absorbent open cell foam that carries the vasoconstrictor and a perimeter portion 662 made from a closed cell foam that is generally impervious to the vasoconstrictor and can provide both a barrier and seal that is positioned to direct vasoconstrictor up into the cervical opening during use. When the foam member 660 abuts the cervix, a compressive force is needed to squeeze or compress the foam member to force the vasoconstrictor out of the foam into the cervical canal and into the uterus. In the embodiment of FIG. 12D, the foam member 660 can be compressed or squeezed by urging the shaft 652 distally when the foam member abuts tissue at the end of the vaginal canal. The shaft indents the foam which squeezes the directed vasoconstrictor into the cervical canal and into the cervix. Other methods of mechanically compressing the foam member can be utilized. This compressive force acts in the same way as squeezing a saturated sponge and with the closed cell foam directing the flow, the vasoconstrictor is forced into the cervical canal and into the uterus. The shaft 652 can include a reservoir to carry the vasoconstrictor within a reservoir housing or multiple reservoirs within the reservoir housing. In some embodiments the foam member 660 can be preloaded with vasoconstrictor and/or the foam member may reside in a chamber containing additional vasoconstrictor so that as the foam member expands, the additional vasoconstrictor is absorbed to keep the foam member generally saturated with vasoconstrictor.

Now referring to FIG. 12E, one alternative expandable foam member 660 is illustrated. A cup like structure of closed cell foam defines the exterior surface of the foam member. This can be made from closed cell foam or alternatively an impervious non-foam polymer layer (with prior embodiments using closed cell foam, an impervious non-foam polymer disclosed here can be utilized instead). Open cell foam containing the vasoconstrictor can form a bulbous member which can be pressed via the shaft 656 against the cervix to compress the foam and release vasoconstrictor. The cup-like structure of closed cell foam or impervous polymer directs the flow of vasoconstrictor into the uterine canal and uterus.

It should be understood that this disclosure is, in many respects, only illustrative. Changes may be made in details, particularly in matters of shape, size, and arrangement of steps without exceeding the scope of the invention. The invention's scope is, of course, defined in the claims hereinafter appended.

We claim:

1. A method for inhibiting uterine bleeding comprising intrautero administration of a therapeutically effective amount of a composition comprising a vasoconstrictor to an individual in need thereof.

2. The method of claim 1, wherein the composition is administered topically intrautero and the therapeutically effective amount of composition comprises sufficient composition to effect administration of about 0.75 mg to about 3 mg vasoconstrictor.

3. The method of claim 1, wherein the vasoconstrictor is an alpha-adrenergic agonist.

4. The method of claim 2, wherein the vasoconstrictor is oxymetazoline.

5. The method of claim 1, wherein the composition further includes a pharmaceutically acceptable fluid-based carrier.

6. The method of claim 5, wherein the vasoconstrictor is oxymetazoline, the composition contains about 0.02% to about 0.08% oxymetazoline, and the therapeutically effective amount is about 1 cc to about 5 cc of the composition.

7. The method of claim 1, wherein the composition further includes a pharmaceutically acceptable time-release carrier.

8. A topical vasoconstrictor delivery system for the uterus comprising: (a) a catheter having a distal end, a proximal end, a distal portion, a proximal portion, and a lumen therethrough, the distal portion sized for passage through the vaginal canal, through the uterine cervix and into the uterus with the proximal portion extending at least into the vaginal canal when the distal portion is positioned within the uterus; and, (b) a means for retaining the distal portion in position within the uterus.

9. The system of claim 8, wherein the means for retaining the distal portion in position within the uterus is a t-shaped distal end.

10. The system of claim 8, wherein the means for retaining the distal portion in position within the uterus is a suture attachment to the vaginal wall on the proximal portion of the catheter.

11. The system of claim 8, wherein the means for retaining the distal portion in position within the uterus is a ring on the proximal portion configured and arranged for disposition within the fornix area when the distal portion is positioned within the uterus.

12. The system of claim 8, wherein the means for retaining the distal portion in position within the uterus is a coiled segment of the proximal portion of the catheter configured and arranged for disposition within the fornix area when the distal portion is positioned within the uterus.

13. The system of claim 8, wherein the catheter is configured and arranged so that the proximal end is positioned within the vaginal canal when the distal portion is positioned within the uterus, and a docking port is provided on the proximal end for receiving an injection assembly such that the injection assembly is in fluid communication with the lumen.

14. An applicator assembly for delivering a topical vasoconstrictor into the uterus comprising: (a) a shaft having a proximal end, a distal end, a proximal portion and a distal portion, the distal portion including an expandable member, the expandable member having a first unexpanded profile for facilitating insertion of the distal portion of the shaft in the vaginal canal, and a second expanded profile wherein the expandable member abuts at least a portion of the cervix to create a fluid path from the expandable member into the cervical canal; and (b) means for transferring the topical vasoconstrictor through the cervical canal into the uterus.

15. The applicator assembly of claim 14 wherein the expandable member includes an expandable positioning ring, the expandable positioning ring having a first unexpanded profile for facilitating insertion of the distal portion of the shaft in the vaginal canal, and a second expanded profile wherein the ring has a bore defining a transverse central axis and is configured and arranged for disposition within the fornix area in the upper vaginal canal with the bore of the ring generally aligned with the lumen of the uterine cervix and the means for transferring topical vasoconstrictor includes an injection port configured and arranged for directing and injecting a fluid through the bore of the ring, through the opening of the uterine cervix and into the uterus.

16. The applicator assembly of claim 14 wherein the expandable member includes a foam member in a compressed state in the first unexpanded position and is in a uncompressed state in the expanded second position wherein the foam abuts at least a portion of the cervix and the means for transferring topical vasoconstrictor includes a compressive force member that squeezes the foam member to reduce its size and force vasoconstrictor therefrom through the opening of the uterine cervix and into the uterus.

17. The applicator assembly of claim 16, wherein the foam member is pre-loaded with vasoconstrictor.

18. The applicator assembly of claim 16 wherein the shaft includes a reservoir containing the vasoconstrictor in fluid communication with the foam member and the foam member absorbs the vasoconstrictor from the reservoir as it expands to become generally saturated therewith in the expanded position.

19. The applicator assembly of claim 16 wherein the foam member comprises a combination of open cell and closed cell foam such that the closed cell foam is generally impervious to the vasoconstrictor and is positioned around a portion of the expanded foam member to direct the flow of vasoconstrictor toward the cervix when the force member squeezes the foam member.

20. The applicator assembly of claim 19 wherein the closed cell foam is positioned to form a seal around the cervix in the area of the fornix.

21. The applicator assembly of claim 16 wherein the compressive force member includes an expandable balloon that urges the foam member toward the cervix in an expanded state.

22. The applicator assembly of claim 16 wherein the compressive force member is a distal portion of the shaft that is urged distally into the expanded foam member to squeeze the foam member.

23. The applicator assembly of claim 15, wherein the shaft further includes multiple reservoirs and a plunger assembly, wherein an α reservoir is in fluid communication with the expandable positioning ring and includes an inflation fluid suitable for expanding the expandable positioning ring upon a first selected actuation of the plunger assembly, and a β reservoir is in fluid communication with the injection port and includes a vasoconstrictor for injection into a uterus through the injection port upon a second selected actuation of the plunger assembly.
